(19) United States
(12) Patent Application Publication
(10) Pub. No.: US 2023/0287051 A1
(43) Pub. Date: Sep. 14, 2023

(54) INHIBITORS OF COMPLEMENT FACTOR C3 AND THEIR MEDICAL USES

(71) Applicant: ZP SPV 3 K/S, Søborg (DK)

(72) Inventors: Anne Pernille Tofteng SHELTON, Søborg (DK); Henrik Fischer MUNCH, Søborg (DK); Rasmus LETH, Søborg (DK)

(21) Appl. No.: 18/016,490

(22) PCT Filed: Jul. 15, 2021
(86) PCT No.: PCT/EP2021/069798

§ 371 Date: Jan. 17, 2023

(30) Foreign Application Priority Data
Jul. 16, 2020 (EP) 20186297.6

(51) Int. Cl. C07K 7/08; A61P 37/06; C07K 14/47

(52) U.S. Cl. CPC C07K 7/08; A61P 37/06; C07K 14/472

(57) ABSTRACT

Compstatin analogues having improved physicochemical properties, such as increased stability and/or solubility as compared to the 13 amino acid compstatin peptide are described, in particular compstatin analogues that additionally possess useful binding and complement-inhibiting activity. These analogues have an alkylene bridge between sulphur atoms of cysteine residues and include variants with an isoleucine residue at position 3 in place of the wild type valine residue, which provides compstatin peptides with improved binding and complement-inhibiting activity and also enables the introduction of other modifications, for example modifications that are capable of increasing stability, such as the introduction of lysine or serine at position 11.

### FIELD OF THE INVENTION

The present invention relates to inhibiting activation of the complement cascade in the body, and more particularly to compstatin analogues that are capable of binding to C3 protein and inhibiting complement activation. The present invention also relates to the medical uses of the compstatin analogues, in particular for the treatment of conditions characterized by unwanted activation of the complement cascade, such as autoimmune and inflammatory diseases.

### BACKGROUND

The human complement system is a powerful player in the defense against pathogenic organisms and the mediation of immune responses. Complement can be activated through three different pathways: the classical, lectin and alternative pathways. The major activation event that is shared by all three pathways is the proteolytic cleavage of the central protein of the complement system, C3, into its activation products C3a and C3b by C3 convertases. Generation of these fragments leads to the opsonization of pathogenic cells by C3b and iC3b, a process that renders them susceptible to phagocytosis or clearance, and to the activation of immune cells through an interaction with complement. Deposition of C3b on target cells also induces the formation of new convertase complexes and thereby initiates a self-amplification loop. An ensemble of plasma and cell surface-bound proteins carefully regulates complement activation to prevent host cells from self-attack by the complement cascade. However, excessive activation or inappropriate regulation of complement can lead to a number of pathologic conditions, ranging from autoimmune to inflammatory diseases. The development of therapeutic complement inhibitors is therefore highly desirable. In this context, C3 and C3b have emerged as promising targets because their central role in the cascade allows for the simultaneous inhibition of the initiation, amplification, and downstream activation of complement.

In view of the therapeutic potential, it remains a problem in the art to further optimize inhibitors of complement factor C3, for example, to achieve an even greater activity and/or to modulate pharmacokinetic properties, such as increased half-life in vivo and/or physicochemical properties such as increased stability and/or solubility.

### SUMMARY

Broadly, the present invention relates to compstatin analogues having an alkylene bridge between sulphur atoms of cysteine residues instead of the disulphide bond found in compstatin. These compstatin analogues have improved physicochemical stability compared to compstatin such as increased stability and/or solubility. Amongst other advantages, it is believed that this may provide improvements in stability (e.g. physical or chemical stability) as compared to equivalent molecules containing disulphide bonds at the corresponding positions. These compstatin analogues may additionally possess improved binding and complement-inhibiting activity as compared to the 13 amino acid compstatin peptide (ICVVQDWGHHRCT (cyclic C2-C12), especially in vivo, as the increased stability may compensate for any reduction in absolute potency resulting from the incorporation of the alkylene bridge instead of a disulphide bond. Introducing such an alkylene binding (bridge) between cysteine residues in positions 2 and 12, for example through use of a thioacetal linkage (e.g. methylene thioacetal) thus improves the overall physicochemical properties for compstatin analogues.

The alkylene bridge introduces (an) additional aliphatic carbon(s) between the two sulphur atoms compared to the disulphide bridge. The alkylene bridge is suitably C₁₋₃alkylene, which may be optionally substituted. The preferred bridge is a C₁-alkylene between the two sulphur atoms, preferably a methylene. In other words, preferably there is a methylene thioacetal linkage (—S—CH₂—S—) between the cysteine residues. The addition of a methylene moiety makes the bridge approximately 1.6 Ångström longer in length and introduces additional degrees of freedom. From molecular dynamics simulations we observe that compstatin analogs with a thioacetal bridge can maintain similar secondary structure found in crystal structure of compstatin bound to C3 (pdb-code: 2QKI). From additional molecular dynamics simulations we have seen that compstatin analogs with a thioacetal bridge can maintain the same intermolecular interactions with C3 as seen for compstatin. During the same simulations we observe an aliphatic-pi stacking interaction between the aliphatic beta-carbon of cysteine 12 and the aromatic sidechain of tryptophan 4. Interestingly, this interaction is also found in the crystal structure of compstatin bound to C3 (pdb-code: 2QKI). Without wishing to be bound by theory, these findings suggest that a thioacetal linkage can be introduced in compstatin analogs while maintaining the same inter- and intramolecular interactions with C3 as seen for compstatin.

Accordingly, in one aspect, the present invention provides a compstatin analogue represented by Formula I:

Y1-R1-X1-C-X3-X4-Q-X6-W-X8-X9-H-X11-C-X13-R2-Y2 (I)

wherein:
Y1 is hydrogen, acetyl or a lipophilic group ϕ;
X1 is I, Y, F or Sar;
X3 is I or V;
X4 is W, F, V, Y, 1-Me-Trp, D-Trp, N-Me-Trp, 1-For-Trp, 1-Nal, 2-Nal, 5MeTrp, Bpa or 2Igl;
X6 is E or D;
X8 is G or Sar;
X9 is H, A, E, D, K, R or S;
X11 is R, K or S;
X13 is T, S, E, F, H, K, Sar, G, I, D, N-Me-Ile or N-Me-Thr;
Y2 is NH₂, OH or a lipophilic group ϕ;
R1 is absent or is a sequence of 1 to 6 amino acid residues selected from A, E, G, L, K, F, P, S, T, W, R, Q, Y, V or Sar, or a corresponding D form thereof; and
R2 is absent or is a sequence of 1 to 6 amino acid residues selected from A, E, G, L, K, F, P, S, T, W, R, V, 8-Amino-3,6-dioxaoctanoyl (Peg3), Sar, γGlu, or a corresponding D form thereof;
wherein the compstatin analogue optionally comprises at least one lipophilic group ϕ covalently linked to the side chain of one or more amino acid residues; and
wherein said compstatin analogue has a C₁₋₃alkylene bridge between the sulphur atoms of the cysteine residues at positions 2 and 12;
or a pharmaceutically acceptable salt or solvate thereof.

Introducing an isoleucine residue at position 3 in place of the wild type valine residue, for example, was found to lead to compstatin peptides with improved binding and complement-inhibiting activity. The introduction of isoleucine at position 3 may also enable the introduction of other modifications that are capable of, for example, increasing stability and/or solubility, such as the introduction of lysine or serine at position 11 and replacement of Thr at position 13 with Ser, Glu, Sar or Ile. Preferred compstatin peptides including one or more of these modifications have improved solubility and/or activity, for example as compared to the compstatin (1-13) peptide (ICVVQDWGHHRCT (having a disulphide bond between C2 and C12) or the known compstatin analogue Cp40.

Accordingly, in another aspect, the present invention provides a compstatin analogue represented by Formula II:

Y1-R1-X1-C-I-X4-Q-X6-W-X8-X9-H-X11-C-X13-R2-Y2 (II)

wherein:
Y1 is hydrogen, acetyl or a lipophilic group ϕ;
X1 is I, Y, F or Sar;
X4 is W, F, V, Y, 1-Me-Trp, D-Trp, N-Me-Trp, 1-For-Trp, 1-Nal, 2-Nal, 5MeTrp, Bpa or 2Igl;
X6 is E or D;
X8 is G or Sar;
X9 is H, A, E, D, K, R or S;
X11 is R, K or S;
X13 is T, S, E, F, H, K, Sar, G, I, D, N-Me-Ile or N-Me-Thr;
Y2 is NH₂ or OH;
R1 is absent or is a sequence of 1 to 6 amino acid residues selected from A, E, G, L, K, F, P, S, T, W, R, Q, Y, V or Sar, or a corresponding D form thereof; and
R2 is absent or is a sequence of 1 to 6 amino acid residues selected from A, E, G, L, K, F, P, S, T, W, R, V, 8-Amino-3,6-dioxaoctanoyl (Peg3), Sar, γGlu, or a corresponding D form thereof;
wherein the compstatin analogue optionally comprises at least one lipophilic group ϕ covalently linked to the side chain of one or more amino acid residues; and
wherein said compstatin analogue has a C₁₋₃alkylene bridge between sulphur atoms of the cysteine residues at positions 2 and 12;
or a pharmaceutically acceptable salt or solvate thereof.

In some embodiments of Formulae I and II:
X1 is I, Y or F;
X4 is W, Y, 1-Me-Trp, 1-Nal, 2-Nal;
X6 is E or D;
X8 is G or Sar;
X9 is A or E;
X11 is R or K; and
X13 is T, S, E, F, H, K, Sar, G, I, D, N-Me-Ile or N-Me-Thr.

The present invention further provides a compstatin analogue represented by Formula III:

Y1-R1-F-C-I-1-Me-Trp-Q-X6-W-X8-E-H-R-C-X13-R2-Y2 (III)

wherein:
Y1 is hydrogen, acetyl or a lipophilic group 0;
X6 is E or D;
X8 is G or Sar;
X13 is T or Sar;
Y2 is NH₂, OH or a lipophilic group ϕ;
R1 is absent or is a sequence of 1 to 6 amino acid residues selected from A, E, G, L, K, F, P, S, T, W, R, Q, Y, V or Sar, or a corresponding D form thereof; and
R2 is absent or is a sequence of 1 to 6 amino acid residues selected from A, E, G, L, K, F, P, S, T, W, R, V, 8-Amino-3,6-dioxaoctanoyl (Peg3), Sar, γGlu or a corresponding D form thereof;
wherein the compstatin analogue optionally comprises at least one lipophilic group ϕ covalently linked to the side chain of one or more amino acid residues; and
wherein said compstatin analogue has a C₁₋₃alkylene bridge between the sulphur atoms of the cysteine residues at positions 2 and 12;
or a pharmaceutically acceptable salt or solvate thereof.

Examples of sequences for the group R1 include:
SE, EGSA, GE, E, {d}Y, EGSE, KSGE, EQEV, ESQV, ESEQV, SEQA, SKQE, EGESG, GQSA, ESGV and YEQA.

Without wishing to be bound by theory, structural considerations suggest that R1 groups including a glutamine (Q) residue may interact particularly well with C3, resulting in increased potency of complement inhibition. This may help to compensate for any reduction in potency resulting from the alkylene linkage between the cysteine side chains, as compared to a disulphide linkage.

Examples of sequences for the group R2 include:
GAES, EGE[Peg3][Peg3]-K*, EK[γGlu]-K*,
EGA-K*, EGE[Peg3]ES-K*,
EAE[Peg3][Peg3]-K*, E[Peg3][Peg3]-K*, EA[Peg3][Peg3]-K*,GAES[Peg3][Peg3]-K* and EGE,
wherein * indicates that the amino acid residue bears a lipophilic group ϕ covalently linked to its side chain.

A lipophilic group ϕ may be covalently linked to the side chain of one or more of the residues in R2, especially to the side chain of a lysine residue. X* indicates that the amino acid residue X bears a lipophilic group ϕ covalently linked to its side chain. It may be desirable that the residue bearing ϕ is at the C-terminus of R2, e.g. a Lys residue at the C-terminus of R2.

The peptide backbone of the compstatin analogue (i.e. excluding the Y1 and Y2 groups) may be represented by the formula:

| | |
|---|---|
| 1; 31 | SEF[C(x)]I[1-Me-Trp]QDWGEHR[C(x)]TGAES |
| 2 | SEF[C(x)]I[1-Me-Trp]QDWGEHR[C(x)][Sar]EGE[Peg3][Peg3]- [K*] |
| 3 | EGSAY[C(x)]I[1-Me-Trp]QDWGEHR[C(x)][Sar]EK[γGlu]-[K*] |
| 4 | Ac-SEF[C(x)]I[1-Me-Trp]QDWGEHR[C(x)][Sar]EGA-[K*] |
| 5 | SEF[C(x)]I[1-Me-Trp]QDWGEHR[C(x)]TEGE[Peg3]ES-[K*] |
| 6 | GEF[C(x)]I[1-Me-Trp]QDWGEHR[C(x)][Sar]EAE[Peg3][Peg3]-[K*] |
| 7 | SEF[C(x)]I[1-Me-Trp]QDW[Sar]EHR[C(x)][Sar]E[Peg3][Peg3]-[K*] |
| 8 | SEF[C(x)]I[1-Me-Trp]QDW[Sar]EHR[C(x)][Sar]E[Peg3][Peg3]-[K*] |
| 9 | SEF[C(x)]I[1-Me-Trp]QDW[Sar]EHR[C(x)]TEGE[Peg3][Peg3]-[K*] |
| 10 | SEF[C(x)]I[1-Me-Trp]QDW[Sar]EHR[C(x)]TE[Peg3][Peg3]-[K*] |
| 11 | SEF[C(x)]I[1-Me-Trp]QDW[Sar]EHR[C(x)]TEGE[Peg3]ES-[K*] |
| 12 | EF[C(x)]I[1-Me-Trp]QDW[Sar]EHR[C(x)]TEA[Peg3][Peg3]-[K*] |
| 13 | SEF[C(x)]I[1-Me-Trp]QDW[Sar]AHR[C(x)]TEGE[Peg3]ES-[K*] |
| 14 | SEF[C(x)]I[1-Me-Trp]QDWGEHR[C(x)]TGAES[Peg3][Peg3]-[K*] |
| 15 | {d}YI[C(x)]I[1-Me-Trp]QDW[Sar]EHR[C(x)]TEGE[Peg3]ES-[K*] |
| 16 | SEF[C(x)]IWQDW[Sar]EHR[C(x)]TEGE[Peg3]ES-[K*] |
| 17 | SEF[C(x)]IYQDW[Sar]EHR[C(x)]TEGE[Peg3]ES-[K*] |
| 18 | SEY[C(x)]I[1-Me-Trp]QDW[Sar]EHR[C(x)]TEGE[Peg3]ES-[K*] |
| 19 | EGSEF[C(x)]I[1-Me-Trp]QDW[Sar]EHR[C(x)]TEGE |
| 20 | EGSEF[C(x)]I[1-Me-Trp]QDW[Sar]EHR[C(x)]TEGE[Peg3]ES-[K*] |
| 21 | KSGEF[C(x)]I[1-Me-Trp]QDW[Sar]EHR[C(x)]TEGE[Peg3][Peg3]-[K*] |
| 22 | EQEVF[C(x)]I[1-Me-Trp]QDW[Sar]EHR[C(x)]TEGE[Peg3][Peg3]-[K*] |
| 23 | ESQVF[C(x)]I[1-Me-Trp]QDW[Sar]EHR[C(x)]TEGE[Peg3][Peg3]-[K*] |
| 24 | ESEQVF[C(x)]I[1-Me-Trp]QDW[Sar]EHR[C(x)]TEGE[Peg3][Peg3]-[K*] |
| 25 | SEQAF[C(x)]I[1-Me-Trp]QDW[Sar]EHR[C(x)]TEGE[Peg3][Peg3]-[K*] |
| 26 | SKQEF[C(x)]I[1-Me-Trp]QDW[Sar]EHR[C(x)]TEGE[Peg3][Peg3]-[K*] |
| 27 | EGESGF[C(x)]I[1-Me-Trp]QDW[Sar]EHR[C(x)]TEGE[Peg3][Peg3]-[K*] |
| 28 | GQSAF[C(x)]I[1-Me-Trp]QDW[Sar]EHR[C(x)]TEGE[Peg3][Peg3]-[K*] |
| 29 | ESGVF[C(x)]I[1-Me-Trp]QDW[Sar]EHR[C(x)]TEGE[Peg3][Peg3]-[K*] |
| 30 | YEQAF[C(x)]I[1-Me-Trp]QDW[Sar]EHR[C(x)]TEGE[Peg3]ES-[K*] |

where [C(x)] indicates pairs of cysteine residues having an alkylene (e.g. methylene (x = 1), ethylene(x = 2) or propylene (x = 3)) bridge between the sulphur atoms of their side chains, and where * indicates that the amino acid residue bears a lipophilic group ϕ covalently linked to its side chain.

The peptide backbone of the compstatin analogue (i.e. excluding the Y1 and Y2 groups) may be represented by the formula:

| | |
|---|---|
| 1 | SEF[C(1)]I[1-Me-Trp]QDWGEHR[C(1)]TGAES |
| 2 | SEF[C(1)]I[1-Me-Trp]QDWGEHR[C(1)][Sar]EGE[Peg3][Peg3]- [K*] |
| 3 | EGSAY[C(1)]I[1-Me-Trp]QDWGEHR[C(1)][Sar]EK[γGlu]-[K*] |
| 4 | Ac-SEF[C(1)]I[1-Me-Trp]QDWGEHR[C(1)][Sar]EGA-[K*] |
| 5 | SEF[C(1)]I[1-Me-Trp]QDWGEHR[C(1)]TEGE[Peg3]ES-[K*] |
| 6 | GEF[C(1)]I[1-Me-Trp]QDWGEHR[C(1)][Sar]EAE[Peg3][Peg3]-[K*] |
| 7 | SEF[C(1)]I[1-Me-Trp]QDW[Sar]EHR[C(1)][Sar]E[Peg3][Peg3]-[K*] |
| 8 | SEF[C(1)]I[1-Me-Trp]QDW[Sar]EHR[C(1)][Sar]E[Peg3][Peg3]-[K*] |
| 9 | SEF[C(1)]I[1-Me-Trp]QDW[Sar]EHR[C(1)]TEGE[Peg3][Peg3]-[K*] |
| 10 | SEF[C(1)]I[1-Me-Trp]QDW[Sar]EHR[C(1)]TE[Peg3][Peg3]-[K*] |
| 11 | SEF[C(1)]I[1-Me-Trp]QDW[Sar]EHR[C(1)]TEGE[Peg3]ES-[K*] |
| 12 | EF[C(1)]I[1-Me-Trp]QDW[Sar]EHR[C(1)]TEA[Peg3][Peg3]-[K*] |
| 13 | SEF[C(1)]I[1-Me-Trp]QDW[Sar]AHR[C(1)]TEGE[Peg3]ES-[K*] |
| 14 | SEF[C(1)]I[1-Me-Trp]QDWGEHR[C(1)]TGAES[Peg3][Peg3]-[K*] |
| 15 | {d}YI[C(1)]I[1-Me-Trp]QDW[Sar]EHR[C(1)]TEGE[Peg3]ES-[K*] |
| 16 | SEF[C(1)]IWQDW[Sar]EHR[C(1)]TEGE[Peg3]ES-[K*] |
| 17 | SEF[C(1)]IYQDW[Sar]EHR[C(1)]TEGE[Peg3]ES-[K*] |
| 18 | SEY[C(1)]I[1-Me-Trp]QDW[Sar]EHR[C(1)]TEGE[Peg3]ES-[K*] |
| 19 | EGSEF[C(1)]I[1-Me-Trp]QDW[Sar]EHR[C(1)]TEGE |
| 20 | EGSEF[C(1)]I[1-Me-Trp]QDW[Sar]EHR[C(1)]TEGE[Peg3]ES-[K*] |
| 21 | KSGEF[C(1)]I[1-Me-Trp]QDW[Sar]EHR[C(1)]TEGE[Peg3][Peg3]-[K*] |
| 22 | EQEVF[C(1)]I[1-Me-Trp]QDW[Sar]EHR[C(1)]TEGE[Peg3][Peg3]-[K*] |
| 23 | ESQVF[C(1)]I[1-Me-Trp]QDW[Sar]EHR[C(1)]TEGE[Peg3][Peg3]-[K*] |
| 24 | ESEQVF[C(1)]I[1-Me-Trp]QDW[Sar]EHR[C(1)]TEGE[Peg3][Peg3]-[K*] |
| 25 | SEQAF[C(1)]I[1-Me-Trp]QDW[Sar]EHR[C(1)]TEGE[Peg3][Peg3]-[K*] |
| 26 | SKQEF[C(1)]I[1-Me-Trp]QDW[Sar]EHR[C(1)]TEGE[Peg3][Peg3]-[K*] |
| 27 | EGESGF[C(1)]I[1-Me-Trp]QDW[Sar]EHR[C(1)]TEGE[Peg3][Peg3]-[K*] |
| 28 | GQSAF[C(1)]I[1-Me-Trp]QDW[Sar]EHR[C(1)]TEGE[Peg3][Peg3]-[K*] |
| 29 | ESGVF[C(1)]I[1-Me-Trp]QDW[Sar]EHR[C(1)]TEGE[Peg3][Peg3]-[K*] |
| 30 | YEQAF[C(1)]I[1-Me-Trp]QDW[Sar]EHR[C(1)]TEGE[Peg3]ES-[K*] |
| 31 | SEFC(2)I[1-Me-Trp]QDWGEHRC(2)TGAES |

where [C(1)] indicates pairs of cysteine residues having a methylene bridging group between the sulphur atoms of their side chains, where [C(2)] indicates pairs of cysteine residues having an ethylene bridging group between the sulphur atoms of their side chains, and where * indicates that the amino acid residue bears a lipophilic group ϕ covalently linked to its side chain.

The peptide backbone of the compstatin analogue (i.e. excluding the Y1 and Y2 groups) may be represented by the formula:

| | |
|---|---|
| 1, 31 | SEF[C(x)]I[1-Me-Trp]QDWGEHR[C(x)]TGAES |
| 2 | SEF[C(x)]I[1-Me-Trp]QDWGEHR[C(x)][Sar]EGE[Peg3][Peg3]-K([17-Carboxy- |
| | heptadecanoyl][γGlu]G[γGlu]) |
| 3 | EGSAY[C(x)]I[1-Me-Trp]QDWGEHR[C(x)][Sar]EK[γGlu]-K([17-Carboxy- |
| | heptadecanoyl][γGlu][Peg3][Peg3]) |
| 4 | Ac-SEF[C(x)]I[1-Me-Trp]QDWGEHR[C(x)][Sar]EGA-K([17-Carboxy- |
| | heptadecanoyl]-[γGlu]G[Peg3][γGlu][Peg3]) |
| 5 | SEF[C(x)]I[1-Me-Trp]QDWGEHR[C(x)]TEGE[Peg3]ES-K([17-Carboxy- |
| | heptadecanoyl]-[γGlu]) |
| 6 | GEF[C(x)]I[1-Me-Trp]QDWGEHR[C(x)][Sar]EAE[Peg3][Peg3]-K([17-Carboxy- |
| | heptadecanoyl][γGlu]G[γGlu]) |
| 7 | SEF[C(x)]I[1-Me-Trp]QDW[Sar]EHR[C(x)][Sar]E[Peg3][Peg3]-K([17-Carboxy- |
| | heptadecanoyl][γGlu]G[γGlu]) |
| 8 | SEF[C(x)]I[1-Me-Trp]QDW[Sar]EHR[C(x)][Sar]E[Peg3][Peg3]-K([17-Carboxy- |
| | heptadecanoyl]-[γGlu]G[γGlu]) |
| 9 | SEF[C(x)]I[1-Me-Trp]QDW[Sar]EHR[C(x)]TEGE[Peg3][Peg3]-K([17-Carboxy- |
| | heptadecanoyl][γGlu]G[γGlu]) |
| 10 | SEF[C(1n)]I[1-Me-Trp]QDW[Sar]EHR[C(x)]TE[Peg3][Peg3]-K([17-Carboxy- |
| | heptadecanoyl][γGlu]G[γGlu]) |
| 11 | SEF[C(x)]I[1-Me-Trp]QDW[Sar]EHR[C(x)]TEGE[Peg3]ES-K([17-Carboxy- |
| | heptadecanoyl][γGlu]) |
| 12 | EF[C(x)]I[1-Me-Trp]QDW[Sar]EHR[C(x)]TEA[Peg3][Peg3]-K([17-Carboxy- |
| | heptadecanoyl][γGlu]G[γGlu]) |
| 13 | SEF[C(x)]I[1-Me-Trp]QDW[Sar]AHR[C(x)]TEGE[Peg3]ES-K([17-Carboxy- |
| | heptadecanoyl][γGlu]) |
| 14 | SEF[C(x)]I[1-Me-Trp]QDWGEHR[C(x)]TGAES[Peg3][Peg3]-K([17-Carboxy- |
| | heptadecanoyl][γGlu]G[γGlu]) |
| 15 | {d}YI[C(x)]I[1-Me-Trp]QDW[Sar]EHR[C(x)]TEGE[Peg3]ES-K([17-Carboxy- |
| | heptadecanoyl][γGlu]) |
| 16 | SEF[C(x)]IWQDW[Sar]EHR[C(x)]TEGE[Peg3]ES-K([17-Carboxy- |
| | heptadecanoyl][γGlu]) |
| 17 | SEF[C(x)]IYQDW[Sar]EHR[C(x)]TEGE[Peg3]ES-K([17-Carboxy- |
| | heptadecanoyl][γGlu]) |
| 18 | SEY[C(x)]I[1-Me-Trp]QDW[Sar]EHR[C(x)]TEGE[Peg3]ES-K([17-Carboxy- |
| | heptadecanoyl][γGlu]) |
| 19 | EGSEF[C(x)]I[1-Me-Trp]QDW[Sar]EHR[C(x)]TEGE |
| 20 | EGSEF[C(x)]I[1-Me-Trp]QDW[Sar]EHR[C(x)]TEGE[Peg3]ES-K([17-Carboxy- |
| | heptadecanoyl][γGlu]) |
| 21 | KSGEF[C(x)]I[1-Me-Trp]QDW[Sar]EHR[C(x)]TEGE[Peg3][Peg3]-K([17- |
| | Carboxy-heptadecanoyl][γGlu]G[γGlu]) |
| 22 | EQEVF[C(x)]I[1-Me-Trp]QDW[Sar]EHR[C(x)]TEGE[Peg3][Peg3]-K([17- |
| | Carboxy-heptadecanoyl][γGlu]G[γGlu]) |
| 23 | ESQVF[C(x)]I[1-Me-Trp]QDW[Sar]EHR[C(x)]TEGE[Peg3][Peg3]-K([17- |
| | Carboxy-heptadecanoyl][γGlu]G[γGlu]) |
| 24 | ESEQVF[C(x)]I[1-Me-Trp]QDW[Sar]EHR[C(x)]TEGE[Peg3][Peg3]-K([17- |
| | Carboxy-heptadecanoyl][γGlu]G[γGlu]) |
| 25 | SEQAF[C(x)]I[1-Me-Trp]QDW[Sar]EHR[C(x)]TEGE[Peg3][Peg3]-K([17- |
| | Carboxy-heptadecanoyl][γGlu]G[γGlu]) |
| 26 | SKQEF[C(x)]I[1-Me-Trp]QDW[Sar]EHR[C(x)]TEGE[Peg3][Peg3]-K([17- |
| | Carboxy-heptadecanoyl][γGlu]G[γGlu]) |
| 27 | EGESGF[C(x)]I[1-Me-Trp]QDW[Sar]EHR[C(x)]TEGE[Peg3][Peg3]-K([17- |
| | Carboxy-heptadecanoyl][γGlu]G[γGlu]) |
| 28 | GQSAF[C(x)]I[1-Me-Trp]QDW[Sar]EHR[C(x)]TEGE[Peg3][Peg3]-K([17- |
| | Carboxy-heptadecanoyl][γGlu]G[γGlu]) |
| 29 | ESGVF[C(x)]I[1-Me-Trp]QDW[Sar]EHR[C(x)]TEGE[Peg3][Peg3]-K([17- |
| | Carboxy-heptadecanoyl][γGlu]G[γGlu]) |
| 30 | YEQAF[C(x)]I[1-Me-Trp]QDW[Sar]EHR[C(x)]TEGE[Peg3]ES-K([17-Carboxy- |
| | heptadecanoyl][γGlu]G[γGlu]) |

where [C(x)] indicates pairs of cysteine residues having an alkylene (e.g. methylene, ethylene or propylene) bridge between the sulphur atoms of their side chains, and where * indicates that the amino acid residue bears a lipophilic group ϕ covalently linked to its side chain.

The peptide backbone of the compstatin analogue (i.e. excluding the Y1 and Y2 groups) may be represented by the formula:

| | |
|---|---|
| 1 | SEF[C(1)]I[1-Me-Trp]QDWGEHR[C(1)]TGAES |
| 2 | SEF[C(1)]I[1-Me-Trp]QDWGEHR[C(1)][Sar]EGE[Peg3][Peg3]-K([17-Carboxy- |
| | heptadecanoyl][γGlu]G[γGlu]) |
| 3 | EGSAY[C(1)]I[1-Me-Trp]QDWGEHR[C(1)][Sar]EK[γGlu]-K([17-Carboxy- |
| | heptadecanoyl][γGlu][Peg3][Peg3]) |
| 4 | Ac-SEF[C(1)]I[1-Me-Trp]QDWGEHR[C(1)][Sar]EGA-K([17-Carboxy- |
| | heptadecanoyl]-[γGlu]G[Peg3][γGlu][Peg3]) |
| 5 | SEF[C(1)]I[1-Me-Trp]QDWGEHR[C(1)]TEGE[Peg3]ES-K([17-Carboxy- |
| | heptadecanoyl]-[γGlu]) |
| 6 | GEF[C(1)]I[1-Me-Trp]QDWGEHR[C(1)][Sar]EAE[Peg3][Peg3]-K([17-Carboxy- |
| | heptadecanoyl][γGlu]G[γGlu]) |
| 7 | SEF[C(1)]I[1-Me-Trp]QDW[Sar]EHR[C(1)][Sar]E[Peg3][Peg3]-K([17-Carboxy- |
| | heptadecanoyl][γGlu]G[γGlu]) |
| 8 | SEF[C(1)]I[1-Me-Trp]QDW[Sar]EHR[C(1)][Sar]E[Peg3][Peg3]-K([17-Carboxy- |
| | heptadecanoyl]-[γGlu]G[γGlu]) |
| 9 | SEF[C(1)]I[1-Me-Trp]QDW[Sar]EHR[C(1)]TEGE[Peg3][Peg3]-K([17-Carboxy- |
| | heptadecanoyl][γGlu]G[γGlu]) |
| 10 | SEF[C(1)]I[1-Me-Trp]QDW[Sar]EHR[C(1)]TE[Peg3][Peg3]-K([17-Carboxy- |
| | heptadecanoyl][γGlu]G[γGlu]) |
| 11 | SEF[C(1)]I[1-Me-Trp]QDW[Sar]EHR[C(1)]TEGE[Peg3]ES-K([17-Carboxy- |
| | heptadecanoyl][γGlu]) |
| 12 | EF[C(1)]I[1-Me-Trp]QDW[Sar]EHR[C(1)]TEA[Peg3][Peg3]-K([17-Carboxy- |
| | heptadecanoyl][γGlu]G[γGlu]) |
| 13 | SEF[C(1)]I[1-Me-Trp]QDW[Sar]AHR[C(1)]TEGE[Peg3]ES-K([17-Carboxy- |
| | heptadecanoyl][γGlu]) |
| 14 | SEF[C(1)]I[1-Me-Trp]QDWGEHR[C(1)]TGAES[Peg3][Peg3]-K([17-Carboxy- |
| | heptadecanoyl][γGlu]G[γGlu]) |
| 15 | {d}YI[C(1)]I[1-Me-Trp]QDW[Sar]EHR[C(1)]TEGE[Peg3]ES-K([17-Carboxy- |
| | heptadecanoyl][γGlu]) |
| 16 | SEF[C(1)]IWQDW[Sar]EHR[C(1)]TEGE[Peg3]ES-K([17-Carboxy- |
| | heptadecanoyl][γGlu]) |
| 17 | SEF[C(1)]IYQDW[Sar]EHR[C(1)]TEGE[Peg3]ES-K([17-Carboxy- |
| | heptadecanoyl][γGlu]) |
| 18 | SEY[C(1)]I[1-Me-Trp]QDW[Sar]EHR[C(1)]TEGE[Peg3]ES-K([17-Carboxy- |
| | heptadecanoyl][γGlu]) |
| 19 | EGSEF[C(1)]I[1-Me-Trp]QDW[Sar]EHR[C(1)]TEGE |
| 20 | EGSEF[C(1)]I[1-Me-Trp]QDW[Sar]EHR[C(1)]TEGE[Peg3]ES-K([17-Carboxy- |
| | heptadecanoyl][γGlu]) |
| 21 | KSGEF[C(1)]I[1-Me-Trp]QDW[Sar]EHR[C(1)]TEGE[Peg3][Peg3]-K([17- |
| | Carboxy-heptadecanoyl][γGlu]G[γGlu]) |
| 22 | EQEVF[C(1)]I[1-Me-Trp]QDW[Sar]EHR[C(1)]TEGE[Peg3][Peg3]-K([17- |
| | Carboxy-heptadecanoyl][γGlu]G[γGlu]) |
| 23 | ESQVF[C(1)]I[1-Me-Trp]QDW[Sar]EHR[C(1)]TEGE[Peg3][Peg3]-K([17- |
| | Carboxy-heptadecanoyl][γGlu]G[γGlu]) |
| 24 | ESEQVF[C(1)]I[1-Me-Trp]QDW[Sar]EHR[C(1)]TEGE[Peg3][Peg3]-K([17- |
| | Carboxy-heptadecanoyl][γGlu]G[γGlu]) |
| 25 | SEQAF[C(1)]I[1-Me-Trp]QDW[Sar]EHR[C(1)]TEGE[Peg3][Peg3]-K([17- |
| | Carboxy-heptadecanoyl][γGlu]G[γGlu]) |
| 26 | SKQEF[C(1)]I[1-Me-Trp]QDW[Sar]EHR[C(1)]TEGE[Peg3][Peg3]-K([17- |
| | Carboxy-heptadecanoyl][γGlu]G[γGlu]) |
| 27 | EGESGF[C(1)]I[1-Me-Trp]QDW[Sar]EHR[C(1)]TEGE[Peg3][Peg3]-K([17- |
| | Carboxy-heptadecanoyl][γGlu]G[γGlu]) |
| 28 | GQSAF[C(1)]I[1-Me-Trp]QDW[Sar]EHR[C(1)]TEGE[Peg3][Peg3]-K([17- |
| | Carboxy-heptadecanoyl][γGlu]G[γGlu]) |
| 29 | ESGVF[C(1)]I[1-Me-Trp]QDW[Sar]EHR[C(1)]TEGE[Peg3][Peg3]-K([17- |
| | Carboxy-heptadecanoyl][γGlu]G[γGlu]) |
| 30 | YEQAF[C(1)]I[1-Me-Trp]QDW[Sar]EHR[C(1)]TEGE[Peg3]ES-K([17-Carboxy- |
| | heptadecanoyl][γGlu]G[γGlu]) |
| 31 | SEFC(2)I[1-Me-Trp]QDWGEHRC(2)TGAES |

where [C(1)] indicates pairs of cysteine residues having a methylene bridging group between the sulphur atoms of their side chains, and [C(2)] indicates pairs of cysteine residues having an ethylene bridging group between the sulphur atoms of their side chains.

The compstatin analogue may be represented by the formula:

| | |
|---|---|
| 1 | Ac-SEF[C(1)]I[1-Me-Trp]QDWGEHR[C(1)]TGAES-[NH₂] |
| 2 | Ac-SEF[C(1)]I[1-Me-Trp]QDWGEHR[C(1)][Sar]EGE[Peg3][Peg3]-K([17- |
| | Carboxy-heptadecanoyl][γGlu]G[γGlu])-NH₂ |
| 3 | Ac-EGSAY[C(1)]I[1-Me-Trp]QDWGEHR[C(1)][Sar]EK[γGlu]-K([17-Carboxy- |
| | heptadecanoyl][γGlu][Peg3][Peg3])-NH₂ |
| 4 | Ac-SEF[C(1)]I[1-Me-Trp]QDWGEHR[C(1)][Sar]EGA-K([17-Carboxy- |
| | heptadecanoyl]-[γGlu]G[Peg3][γGlu][Peg3]-NH2 |
| 5 | Ac-SEF[C(1)]I[1-Me-Trp]QDWGEHR[C(1)]TEGE[Peg3]ES-K([17-Carboxy- |
| | heptadecanoyl]-[γGlu]-NH₂ |
| 6 | Ac-GEF[C(1)]I[1-Me-Trp]QDWGEHR[C(1)][Sar]EAE[Peg3][Peg3]-K([17- |
| | Carboxy-heptadecanoyl][γGlu]G[γGlu])-NH₂ |
| 7 | Ac-SEF[C(1)]I[1-Me-Trp]QDW[Sar]EHR[C(1)][Sar]E[Peg3][Peg3]-K([17- |
| | Carboxy-heptadecanoyl][γGlu]G[γGlu])-NH₂ |
| 8 | Ac-SEF[C(1)]I[1-Me-Trp]QDW[Sar]EHR[C(1)][Sar]E[Peg3][Peg3]-K([17- |
| | Carboxy-heptadecanoyl]-[γGlu]G[γGlu]-NH₂ |
| 9 | Ac-SEF[C(1)]I[1-Me-Trp]QDW[Sar]EHR[C(1)]TEGE[Peg3][Peg3]-K([17- |
| | Carboxy-heptadecanoyl][γGlu]G[γGlu])-NH₂ |
| 10 | Ac-SEF[C(1)]I[1-Me-Trp]QDW[Sar]EHR[C(1)]TE[Peg3][Peg3]-K([17-Carboxy- |
| | heptadecanoyl][γGlu]G[γGlu])-NH₂ |
| 11 | Ac-SEF[C(1)]I[1-Me-Trp]QDW[Sar]EHR[C(1)]TEGE[Peg3]ES-K([17-Carboxy- |
| | heptadecanoyl][γGlu])-NH₂ |
| 12 | Ac-EF[C(1)]I[1-Me-Trp]QDW[Sar]EHR[C(1)]TEA[Peg3][Peg3]-K([17-Carboxy- |
| | heptadecanoyl][γGlu]G[γGlu])-NH₂ |
| 13 | Ac-SEF[C(1)]I[1-Me-Trp]QDW[Sar]AHR[C(1)]TEGE[Peg3]ES-K([17-Carboxy- |
| | heptadecanoyl][γGlu])-NH₂ |
| 14 | Ac-SEF[C(1)]I[1-Me-Trp]QDWGEHR[C(1)]TGAES[Peg3][Peg3]-K([17- |
| | Carboxy-heptadecanoyl][γGlu]G[γGlu])-NH₂ |
| 15 | Ac-{d}YI[C(1)]I[1-Me-Trp]QDW[Sar]EHR[C(1)]TEGE[Peg3]ES-K([17-Carboxy- |
| | heptadecanoyl][γGlu])-NH₂ |
| 16 | Ac-SEF[C(1)]IWQDW[Sar]EHR[C(1)]TEGE[Peg3]ES-K([17-Carboxy- |
| | heptadecanoyl][γGlu])-NH₂ |
| 17 | Ac-SEF[C(1)]IYQDW[Sar]EHR[C(1)]TEGE[Peg3]ES-K([17-Carboxy- |
| | heptadecanoyl][γGlu])-NH₂ |
| 18 | Ac-SEY[C(1)]I[1-Me-Trp]QDW[Sar]EHR[C(1)]TEGE[Peg3]ES-K([17-Carboxy- |
| | heptadecanoyl][γGlu])-NH₂ |
| 19 | [15-Carboxy-pentadecanoyl]-EGSEF[C(1)]I[1-Me- |
| | Trp]QDW[Sar]EHR[C(1)]TEGE-[NH₂] |
| 20 | Ac-EGSEF[C(1)]I[1-Me-Trp]QDW[Sar]EHR[C(1)]TEGE[Peg3]ES-K([17- |
| | Carboxy-heptadecanoyl][γGlu])-NH₂ |
| 21 | Ac-KSGEF[C(1)]I[1-Me-Trp]QDW[Sar]EHR[C(1)]TEGE[Peg3][Peg3]-K([17- |
| | Carboxy-heptadecanoyl][γGlu]G[γGlu])-NH₂ |
| 22 | Ac-EQEVF[C(1)][1-Me-Trp]QDW[Sar]EHR[C(1)]TEGE[Peg3][Peg3]-K([17- |
| | Carboxy-heptadecanoyl][γGlu]G[γGlu])-NH₂ |
| 23 | Ac-ESQVF[C(1)]I[1-Me-Trp]QDW[Sar]EHR[C(1)]TEGE[Peg3][Peg3]-K([17- |
| | Carboxy-heptadecanoyl][γGlu]G[γGlu])-NH₂ |
| 24 | Ac-ESEQVF[C(1)]I[1-Me-Trp]QDW[Sar]EHR[C(1)]TEGE[Peg3][Peg3]-K([17- |
| | Carboxy-heptadecanoyl][γGlu]G[γGlu])-NH₂ |
| 25 | Ac-SEQAF[C(1)]I[1-Me-Trp]QDW[Sar]EHR[C(1)]TEGE[Peg3][Peg3]-K([17- |
| | Carboxy-heptadecanoyl][γGlu]G[γGlu])-NH₂ |
| 26 | Ac-SKQEF[C(1)]I[1-Me-Trp]QDW[Sar]EHR[C(1)]TEGE[Peg3][Peg3]-K([17- |
| | Carboxy-heptadecanoyl][γGlu]G[γGlu])-NH₂ |
| 27 | Ac-EGESGF[C(1)]I[1-Me-Trp]QDW[Sar]EHR[C(1)]TEGE[Peg3][Peg3]-K([17- |
| | Carboxy-heptadecanoyl][γGlu]G[γGlu])-NH₂ |
| 28 | Ac-GQSAF[C(1)]I[1-Me-Trp]QDW[Sar]EHR[C(1)]TEGE[Peg3][Peg3]-K([17- |
| | Carboxy-heptadecanoyl][γGlu]G[γGlu])-NH₂ |
| 29 | H-ESGVF[C(1)]I[1-Me-Trp]QDW[Sar]EHR[C(1)]TEGE[Peg3][Peg3]-K([17- |
| | Carboxy-heptadecanoyl][γGlu]G[γGlu])-NH₂ |
| 30 | Ac-YEQAF[C(1)]I[1-Me-Trp]QDW[Sar]EHR[C(1)]TEGE[Peg3]ES-K([17- |
| | Carboxy-heptadecanoyl][γGlu]G[γGlu])-NH₂ |
| 31 | Ac-SEFC(2)I[1-Me-Trp]QDWGEHRC(2)TGAES-NH₂ |

where [C(1)] indicates pairs of cysteine residues having a methylene bridgebetween the sulphur atoms of their side chains (that is, the [C(1)] residues have a —S—CH₂—S— linkage) and [C(2)] indicates pairs of cysteine residues having an ethylene bridging group between the sulphur atoms of their side chains (that is, the [C(2)] residues have a —S—CH₂—CH₂—S— linkage).

In a further aspect, described herein is a composition comprising a compstatin analogue, or a pharmaceutically acceptable salt or solvate thereof, in admixture with a carrier. In some instances, the composition is a pharmaceutical composition and the carrier is a pharmaceutically acceptable carrier.

In a further aspect, described herein is a pharmaceutical composition comprising a compstatin analogue, or a pharmaceutically acceptable salt or solvate thereof, in admixture with a pharmaceutically acceptable carrier, excipient or vehicle.

In a further aspect, described herein is a compstatin analogue for use in therapy.

In a further aspect, described herein is a compstatin analogue for use in a method of inhibiting complement activation. By way of example, inhibiting complement activation includes one or more biological activities selected from (1) binding to C3 protein, (2) binding to C3b protein and/or (3) inhibiting the cleavage of native C3 by C3 convertases. Examples of diseases or conditions that may be treated using the compstatin analogues are discussed below.

In a further aspect, described herein is a compstatin analogue for use in a method of inhibiting complement activation that occurs during cell or organ transplantation.

In a further aspect, described herein is a method of inhibiting complement activation for treating a subject in need thereof, the method comprising administering to the subject a compstatin analogue, thereby inhibiting complement activation in the subject. Examples of diseases or conditions that may be treated using the compstatin analogues are discussed below.

In a further aspect, described herein is an ex vivo method of inhibiting complement activation during extracorporeal shunting of a physiological fluid, the method comprising contacting the physiological fluid with a compstatin analogue, thereby inhibiting complement activation.

In a further aspect, described herein is the use of a compstatin analogue in the preparation of a medicament for inhibiting complement activation. Examples of diseases or conditions that may be treated using the compstatin analogues of the present invention are discussed below.

### DESCRIPTION OF THE FIGURES

FIG. 1

Normalized “ex vivo” activity of the alternative complement pathway over time after administration of a test compound at time 0 to non-human primates. Compounds were given subcutaneously at a dose of 1840 nmol/kg. Complement activity (alternative pathway) was measured using the Wieslab kit. Activity was normalized using the predose (0) sample (set to 100%) and the negative control included in the kit. Normalized activity or average normalized activity and standard deviation is shown. (a) compound 2, compound 5, compound 7 (all one animal per compound) and Cp40 (4 animals); (b) compound 12, compound 20 & comp 25, all with one animal per compound and Cp40 (4 animals).

### DETAILED DESCRIPTION

As used herein, “and/or” is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment and apply equally to all aspects and embodiments that are described.

Various publications, including patents, published applications, technical articles and scholarly articles are cited throughout the specification. Each of these cited publications is incorporated by reference herein, in its entirety.

Unless otherwise defined herein, scientific and technical terms used in this application shall have the meanings that are commonly understood by those of ordinary skill in the art.

Generally, nomenclature used in connection with, and techniques of, chemistry, molecular biology, cell and cancer biology, immunology, microbiology, pharmacology, and protein and nucleic acid chemistry, described herein, are those known and commonly used in the art.

Each embodiment described herein may be taken alone or in combination with one or more other embodiments.

Unless specified otherwise, the following definitions are provided for specific terms that are used in the present written description.

### Definitions

Throughout this specification, the word “comprise,” and grammatical variants thereof, such as “comprises” or “comprising”, will be understood to imply the inclusion of a stated integer or component, or group of integers or components, but not the exclusion of any other integer or component, or group of integers or components.

The singular forms “a,” “an” and “the” include the plurals unless the context clearly dictates otherwise.

The term “including” is used to mean “including but not limited to”. “Including” and “including but not limited to” may be used interchangeably.

The terms “patient,” “subject” and “individual” may be used interchangeably. A subject may be a mammal, including a human or a non-human mammal, such as a non-human primate (e.g., ape, Old World monkey or New World monkey), livestock animal (e.g., bovine or porcine), companion animal (e.g., canine or feline) or laboratory animal such as a rodent (e.g., mouse or rat).

Throughout the present description and claims the conventional three-letter and one-letter codes for naturally occurring amino acids are used, i.e., A (Ala), G (Gly), L (Leu), I (Ile), V (Val), F (Phe), W (Trp), S (Ser), T (Thr), Y (Tyr), N (Asn), Q (Gln), D (Asp), E (Glu), K (Lys), R (Arg), H (His), M (Met), C (Cys) and P (Pro); as well as generally accepted three-letter codes for other α-amino acids, such as norleucine (Nle), sarcosine (Sar), α-aminoisobutyric acid (Aib), 2,3-diaminopropanoic acid (Dap), 2,4-diaminobutanoic acid (Dab) and 2,5-diaminopentanoic acid (ornithine; Orn), 1-methyl-tryptophan (1-Me-Trp, 1Me-Trp or 1MeTrp), 1-formyl-tryptophan (1-For-Trp or 1For-Trp or 1ForTrp), 1-naphathalin (1-Nal or 1Nal), 2-naphathalin (2-Nal or 2Nal), 5-methyl-tryptophan (5-Me-Trp or 5Me-Trp or 5MeTrp), p-Benzoyl-phenylalanine (Bpa) 2-indanylglycine (2Igl or 2-Igl). Other α-amino acids may be shown in square brackets “[ ]” (e.g., “[Nle]”) when used in a general formula or sequence in the present specification, especially when the rest of the formula or sequence is shown using the single letter code. The 20 “naturally occurring” amino acids listed above are those that are encoded by the standard genetic code, and may also be referred to as “proteinogenic” amino acids.

Gamma-Glu and beta-Asp, also referred to as γGlu (γ-Glu) and βAsp (β-Asp) (or isoGlu and isoAsp), refer to glutamate or aspartate participating in peptide bonds via the γ- or β-carboxylic acid respectively (normally regarded as the side chain carboxyl groups), rather than the conventional configuration. Similarly, εLys or isoLys refers to lysine participating in a peptide bond via the epsilon amino group (normally regarded as the side chain amino group) rather than the alpha amino group.

Beta-Ala, also referred to as β-Ala or βAla, refers to 3-aminopropanoic acid.

Peg3 refers to a residue of 8-amino-3,6-dioxaoctanoic acid (also known as {2-[2-aminoethoxy]ethoxy}acetic acid) and Peg4 refers to a residue of 11-amino-3,6,9-trioxaundecanoic acid. The residue may also be denoted [8-Amino-3,6-dioxaoctanoyl].

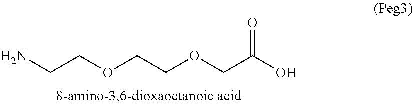 [SMILES: NCCOCCOCC(=O)O]

Unless otherwise specified, amino acid residues in peptides described herein are of the L-configuration. However, in some instances, D-configuration amino acids may be incorporated. In the present context, an amino acid code written with a small letter represents the D-configuration of said amino acid, e.g., “k” represents the D-configuration of lysine (K), or a D-configuration amino acid may be written as (d)X or {d}X, where X is the amino acid, e.g., (d)Y or {d}Y represents the D-configuration of tyrosine (Y).

Cysteine residues shown as “C(x)” indicate that their side-chains participate in a dithioether linkage. That is, they are bridged. Thus there will typically be two such residues in any given molecule. The bridge is an alkylene group linking the sulphur atoms of the cysteine residues. Suitably, the aliphatic group is a short (C₁₋₃-alkylene) moiety, which may be unsubstitued or optionally substituted. Preferably, it is unsubstituted. In some cases, two [C(x)] residues may be bridged by —S—(CH₂)ₙ—S—, where n is 1, 2, or 3 and the sulphur atoms are part of the cysteine residue side chain. Preferably, n is 1 or 2 (that is, methylene or ethylene bridging groups), more preferably n is 1. When n is 1, the linkage may be referred to as a thioacetal. Such a thioacetal may also be referred to in the art as a dithioacetal.

The thioacetal may be a methylene thioacetal. That is, the bridge is a methylene and the two [C(x)] residues are connected by a —S—CH₂—S— linkage. This is typically designated by residues shown as C(1).

The bridging group may be an ethylene bridging group, i.e. the two [C(x)] residues are connected by a —S—CH₂—CH₂—S— linkage. This is typically designated by residues shown as C(2).

The bridging group may be propylene bridging group, i.e. the two [C(x)] residues are connected by a —S—CH₂—CH₂—CH₂—S— linkage. This is typically designated by residues shown as C(3).

Methods of introducing such bridges between cysteine residues will be apparent to the skilled person, and may include nucleophilic substitution of leaving groups by the sulphur atoms of the cysteine residues. For example, a methylene thioacetal linkage can be inserted through double displacement of diiodomethane.

In a similar manner, cysteine residues shown as “C(*)” indicate that their side-chains participate in a disulphide bond.

The terminal groups present at the N- and C-termini of the peptide backbone are designated Y1 and Y2 respectively. Thus Y1 is bonded to the nitrogen atom of the N-terminal amino group and Y2 is bonded to the C-terminal carbonyl carbon atom.

Y1=hydrogen (also indicated as “H—” or “Hy-”) indicates a hydrogen atom, corresponding to the presence of a free primary or secondary amino group at the N-terminus. Y1=acetyl (“Ac”) indicates the presence of an N-terminal secondary acetyl amide group.

Y2=“OH” or “NH₂” indicates the presence of a carboxy (COOH) group or an amido (CONH₂) group at the C-terminus of the molecule.

In some embodiments, Y1 is hydrogen or acetyl, and Y2 is NH₂.

Alternatively one or both of the Y1 and Y2 groups may independently be a lipophilic group ϕ, as described elsewhere in this specification.

### Lipophilic Substituents

The compstatin analogues may bear a lipophilic group, designated ϕ.

The lipophilic group may be covalently linked to the N-terminus and/or the C terminus of the molecule, i.e. Y1 may be ϕ (in place of H or Ac) and/or Y2 may be ϕ (in place of OH or NH₂). Typically only one of Y1 and Y2 is a lipophilic group ϕ, particularly Y1.

Additionally or alternatively, the lipophilic group may be covalently linked to the side chain of an amino acid residue within the analogue. The residue may be part of R1, R2 or the compstatin analogue portion X1-X13 of the molecule. When part of R2, it may be desirable that the residue is the C-terminal residue of R2. Within the compstatin analogue portion X1-X13 of the molecule, position X11 may be particularly suitable.

The lipophilic group ϕ is typically attached via an acyl group. The modification may therefore be termed acylation but can also be refered to as lipidation.

The lipophilic group includes a long chain alkylene group derived from a fatty acid, termed Z¹ herein and referred to as the lipophilic substituent. Without wishing to be bound by theory, it is believed that a lipophilic substituent binds plasma proteins (e.g. albumin) in the blood stream, thus shielding the compounds employed in the context of the invention from enzymatic degradation, and thereby enhancing the half-life of the compounds. The lipophilic substituent may also modulate the potency of the compound.

Z¹ may be attached directly to the amino acid sequence (including the R1 and R2 extensions, or as Y1) or via a spacer Z² as defined herein.

In other words, ϕ may be Z¹— or Z¹—Z²—.

Where Y1 is ϕ, ϕ is preferably Z¹—.

Where the lipophilic group ϕ is linked to an amino acid side chain (i.e. where Y1 is hydrogen or Ac) ϕ may preferably be Z¹—Z²—.

In certain embodiments, only one amino acid side chain is conjugated to a lipophilic substituent. In other embodiments, two amino acid side chains are each conjugated to a lipophilic substituent. In yet further embodiments, three or even more amino acid side chains are each conjugated to a lipophilic substituent. When a compound contains two or more lipophilic substituents, they may be the same or different substituents.

In certain embodiments, only one lipophilic group ϕ is present in the molecule.

The term “conjugated” is used here to describe the covalent attachment of one identifiable chemical moiety to another, and the structural relationship between such moieties. It should not be taken to imply any particular method of synthesis. The one or more spacers Z², when present, are used to provide a spacing between the compound and the lipophilic substituent Z¹.

A lipophilic substituent may be attached to an N-terminal nitrogen, or to an amino acid side chain or to a spacer via an ester, a sulphonyl ester, a thioester, an amide or a sulphonamide. Accordingly, it will be understood that a lipophilic substituent may include an acyl group, a sulphonyl group, an N atom, an O atom or an S atom which forms part of the ester, sulphonyl ester, thioester, amide or sulphonamide.

Suitably, an acyl group in the lipophilic substituent forms part of an amide or ester with the N-terminal nitrogen, or amino acid side chain, or the spacer. The lipophilic substituent may include a hydrocarbon chain having 10 to 24 carbon (C) atoms, e.g. 10 to 22 C atoms, e.g. 10 to 20 C atoms. Preferably, it has at least 11 C atoms, and preferably it has 18 C atoms or fewer. For example, the hydrocarbon chain may contain 12, 13, 14, 15, 16, 17 or 18 carbon atoms. The hydrocarbon chain may be linear or branched and may be saturated or unsaturated.

The hydrocarbon chain may incorporate a phenylene or piperazinylene moiety in its length as, for example, shown below (wherein --- represents the points of attachment within the chain). These groups should be “counted” as 4 carbon atoms in the chain length.

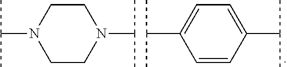

From the discussion above, it will be understood that the hydrocarbon chain may be substituted with a moiety which forms part of the attachment to the amino acid side chain or the spacer, for example an acyl group, a sulphonyl group, an N atom, an O atom or an S atom. Most preferably, the hydrocarbon chain is substituted with an acyl group, and accordingly the hydrocarbon chain may be part of an alkanoyl group, for example a dodecanoyl, 2-butyloctanoyl, tetradecanoyl, hexadecanoyl, heptadecanoyl, octadecanoyl or eicosanoyl group. Alternatively, Z¹ groups are derived from long-chain saturated α,ω-dicarboxylic acids of formula HOOC—(CH₂)₁₂₋₂₂—COOH, preferably from long-chain saturated α,ω-dicarboxylic acids having an even number of carbon atoms in the aliphatic chain.

In other words, Z¹ may be A-C₁₂₋₂₂alkylene-(CO)—, where A is H or —COOH, and wherein the akylene may be linear or branched and may be saturated or unsaturated, and may optionally incorporate a phenylene or piperazinylene moiety in its length.

For example, Z¹ may be:
Dodecanoyl i.e. H—(CH₂)₁₁—(CO)—;
Tetradecanoyl i.e. H—(CH₂)₁₃—(CO)—;
Hexadecanoyl, i.e. H—(CH₂)₁₅—(CO)—;
13-carboxytridecanoyl, i.e. HOOC—(CH₂)₁₂—(CO)—;
15-carboxypentadecanoyl, i.e. HOOC—(CH₂)₁₄—(CO)—;
17-carboxyheptadecanoyl, i.e. HOOC—(CH₂)₁₆—(CO)—;
19-carboxynonadecanoyl, i.e. HOOC—(CH₂)₁₈—(CO)—; or
21-carboxyheneicosanoyl, i.e. HOOC—(CH₂)₂₀—(CO)—

The carboxylic acid, if present, may be replaced by a bioisotere, phosphate or sulfonate. Suitable bioisoteres for carboxylic acids are known in the art and include tetrazole, acylsulfomides, acylhydroxylamine, and squaric acid derivatives.

As mentioned above, the lipophilic substituent Z¹ may be conjugated to the amino acid side chain or N-terminal nitrogen by one or more spacers Z².

When present, the spacer is attached to the lipophilic substituent and to the amino acid side chain or N-terminal nitrogen. The spacer may be attached to the lipophilic substituent and to the amino acid side chain independently by an ester, a sulphonyl ester, a thioester, an amide or a sulphonamide. Accordingly, it may include two moieties independently selected from acyl, sulphonyl, an N atom, an O atom or an S atom. The spacer may consist of a linear C₁₋₁₀ hydrocarbon chain or more preferably a linear C₁₋₅ hydrocarbon chain. Furthermore the spacer can be substituted with one or more substituents selected from C₁₋₆ alkyl, C₁₋₆ alkyl amine, C₁₋₆ alkyl hydroxy and C₁₋₆ alkyl carboxy.

The spacer may be, for example, a residue of any naturally occurring or unnatural amino acid. For example, the spacer may be a residue of Gly, Pro, Ala, Val, Leu, lie, Met, Cys, Phe, Tyr, Trp, His, Lys, Arg, Gln, Asn, Glu, Asp, γ-Glu, β-Asp, ε-Lys, Asp, Ser, Thr, Dapa, Gaba, Aib, β-Ala (i.e., 3-aminopropanoyl), 4-aminobutanoyl, 5-aminopentanoyl, 6-aminohexanoyl, 7-aminoheptanoyl, 8-aminooctanoyl, 9-aminononanoyl, 10-aminodecanoyl, 8-amino-3,6-dioxaoctanoyl. In certain embodiments, the spacer is a residue of Glu, γ-Glu, ε-Lys, β-Ala (i.e., 3-aminopropanoyl), 4-aminobutanoyl, 8-aminooctanoyl or 8-amino-3,6-dioxaoctanoyl (Peg3), 11-amino-3,6,9-trioxaundecanoic acid (Peg4) or (piperazine-1-yl)-carboxylic acid. In the present invention, γGlu and isoGlu are used interchangeably.

Z² is suitably a sequence of 1 to 6 residues of compounds selected from γGlu, βAsp, D, E, K, Orn, S, T, A, βAla, G, P, V, L, I, Y, Q, N, Dapa, Gaba, or Aib, or a corresponding D form thereof, 5-aminopentanoyl, 6-aminohexanoyl, 7-aminoheptanoyl, 8-aminooctanoyl, 9-aminononanoyl, and 10-aminodecanoyl. 8-amino-3,6-dioxaoctanoic acid (Peg3), 11-amino-3,6,9-trioxaundecanoic acid (Peg4) or (piperazine-1-yl)-carboxylic acid.

For example, Z² may be, or may comprise:
[γGlu];
[γGlu][Peg3][Peg3]-;
[(Piperazine-1-yl)-acetyl][Peg3][Peg3];
[γGlu]-G-[γGlu];
[γGlu]-K-[γGlu];
[γGlu]-KG-[γGlu]; or
[γGlu]-G-[Peg3][γGlu][Peg3].

Z² is suitably bound at each side by amide linkage. Other suitable linkages may be used, with the commensurate atom replacement; for example sulfinamide, sulfonamide, or ester linkages or amino, ether, or thioether linkages are envisaged.

In other words, in some aspects the lipophilic group ϕ is Z¹— or Z¹—Z²—; wherein
Z¹ is A-C₁₂₋₂₂alkylene-(CO)—;
where A is H or —COOH, and wherein the akylene may be linear or branched and may be saturated or unsaturated, and may optionally incorporate a phenylene or piperazinylene moiety in its length; and
Z² is a sequence of 1 to 6 of residues of compounds selected from γ-Glu, βAsp, D, E, K, Orn, S, T, A, p-Ala, G, P, V, L, I, Y, Q, N, Dapa, Gaba, or Aib, or a corresponding D form thereof, 5-aminopentanoyl, 6-aminohexanoyl, 7-aminoheptanoyl, 8-aminooctanoyl, 9-aminononanoyl, and 10-aminodecanoyl. 8-amino-3,6-dioxaoctanoic acid (Peg3), 11-amino-3,6,9-trioxaundecanoic acid (Peg4) or (piperazine-1-yl)-carboxylic acid, e.g. a linker selected from
[Glu],
[γGlu][Peg3][Peg3]-;
[(Piperazine-1-yl)-acetyl][Peg3][Peg3];
[γGlu]-G-[γGlu];
[γGlu)-K-[γGlu];
[γGlu]-KG-[γGlu]; and
[γGlu]-G-[Peg3][γGlu][Peg3].

The amino acid side chain to which the lipophilic substituent is conjugated typically includes a carboxy, hydroxyl, thiol, amide or amine group, for forming an ester, a sulphonyl ester, a thioester, an amide, or a sulphonamide with the spacer or lipophilic substituent. An amide linkage may be particularly preferred, and thus the amino acid may be any amino acid having an amine group in its side chain, although it will be clear that side chains having other functional groups are contemplated. Thus, for example, the amino acid side chain may be a side chain of a Glu, Lys or Ser residue. For example, it may be a side chain of a Lys or Glu residue. Where two or more side chains carry a lipophilic substituent, they may be independently selected from those residues.

Typically, the amino acid side chain is a side chain of a Lys residue.

An example of a lipophilic substituent comprising a lipophilic moiety Z¹ and spacer Z² is shown in the formula below:

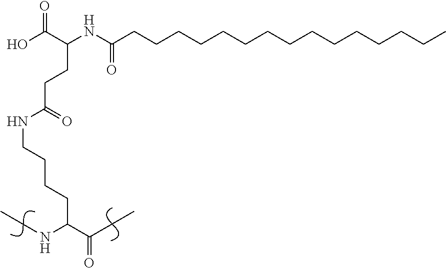 [SMILES: CCCCCCCCCCCCCCCC(=O)NC(CCC(=O)NCCCCC(NCC)C(=O)CC)C(=O)O]

Illustrative structures of lipophilic groups are shown below, where the wavy line indicates the linkage to the peptide (i.e. to the amino acid side chain):

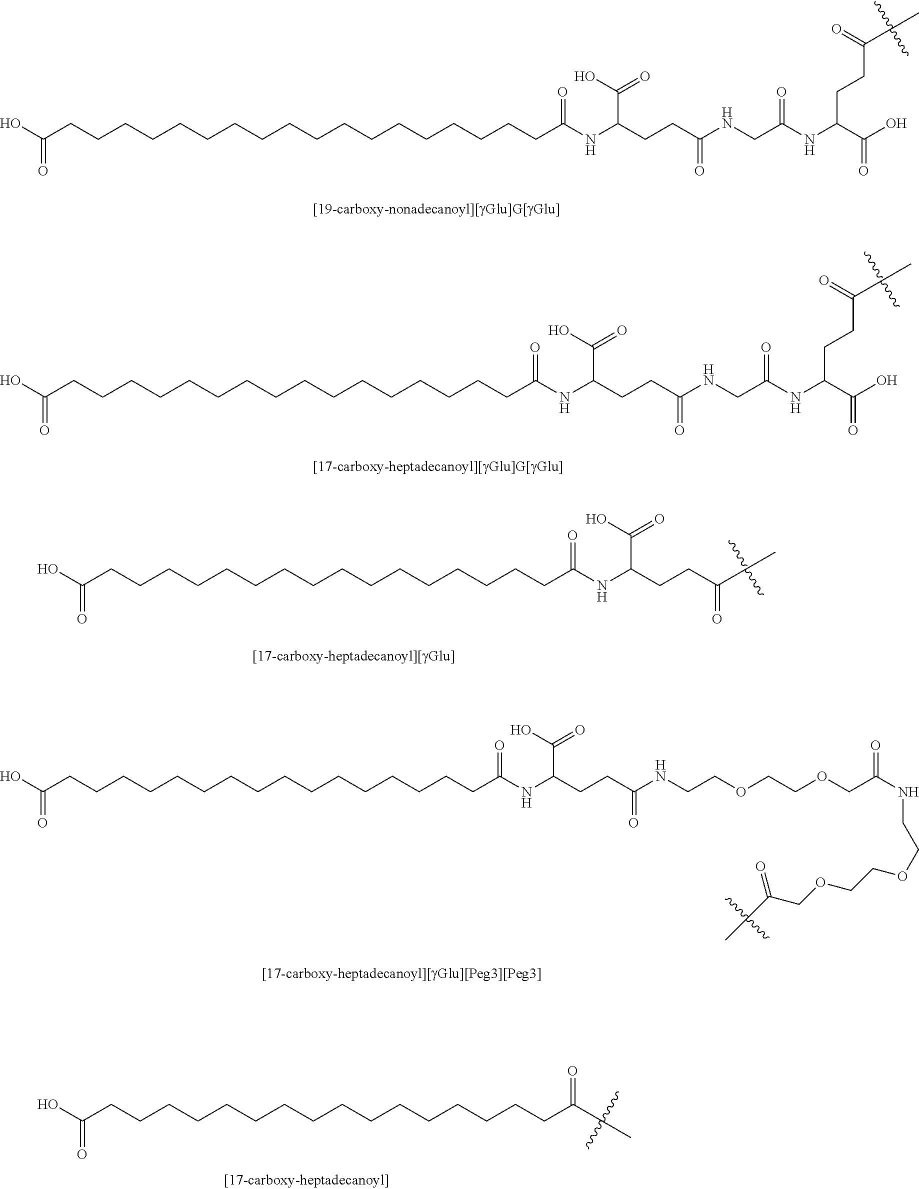 [SMILES: *C(=O)CCC(NC(=O)CNC(=O)CCC(NC(=O)CCCCCCCCCCCCCCCCCCC(=O)O)C(=O)O)C(=O)O ; *C(=O)CCC(NC(=O)CNC(=O)CCC(NC(=O)CCCCCCCCCCCCCCCCC(=O)O)C(=O)O)C(=O)O ; *C(=O)CCC(NC(=O)CCCCCCCCCCCCCCCCC(=O)O)C(=O)O ; *C(=O)COCCOCCNC(=O)COCCOCCNC(=O)CCC(NC(=O)CCCCCCCCCCCCCCCCC(=O)O)C(=O)O ; *C(=O)CCCCCCCCCCCCCCCCC(=O)O]

Various terms relating to the methods and other aspects described herein are used throughout the specification and claims. Such terms are to be given their ordinary meaning in the art unless otherwise indicated. Other specifically defined terms are to be construed in a manner consistent with the definition provided herein. The term “about” as used herein when referring to a measurable value such as an amount, a temporal duration, and the like, is meant to encompass variations of ±20% or ±10%, in some embodiments ±5%, in some embodiments ±1%, and in some embodiments ±0.1% from the specified value, as such variations are appropriate to make and used the disclosed compounds and compositions.

The term “full length compstatin” as used herein refers to a 27 amino acid peptide having the sequence IC(*)VVQDWGHHRC(*)TAGHMANLTSHASAI, wherein C(*) denotes the cysteine residue linked by a disulphide bond. As described above, a truncated form of full length compstatin, the tridecapeptide H-Ile¹-Cys²-Val³-Val⁴-Gln⁵-Asp⁶-Trp⁷-Gly⁸-His⁹-His¹⁰-Arg¹-Cys¹²-Thr¹³-NH₂ linked by a disulphide bond between the cysteine residues at positions 2 and 12 retains the activity of the full length peptide. An N-terminally acetylated version of this tridecapeptide peptide is referred to herein as “Ac-compstatin.”

The term “compstatin analogue” refers to a modified Ac-compstatin comprising one or more substitutions of natural and unnatural amino acids, or amino acid analogs, as well as modifications within or between various amino acids, as described in greater detail herein. A compstatin analogue may comprise about 1, 2, 3, 4 or 5 amino acid modifications relative to Ac-compstatin. A compstatin analogue may comprise 5, 6, 7, 8 or more amino acid modifications relative to Ac-compstatin. A compstatin analogue may comprise about 5, 6, 7 or 8 amino acid modifications relative to Ac-compstatin.

The term “analogue” is frequently used for a protein or peptide in question before it undergoes further chemical modification (derivatisation), and in particular acylation. The product resulting from such a chemical modification (derivatisation) is sometimes referred to as a “derivative” or “acylated analogue.” However, in the context of this application, the term “analogue” designates analogues of Ac-compstatin as well as (the acylated) derivatives of such Ac-compstatin analogues.

When referring to the position of amino acids or analogs within Ac-compstatin or compstatin analogs, the positions are numbered from 1 (Ile in compstatin) to 13 (Thr in compstatin). For example, the Gly residue occupies “position 8.”

The terms “pharmaceutically active” and “biologically active” refer to the ability of the compounds to bind C3 or fragments thereof and inhibit complement activation. The biological activities of compstatin analogs may be measured by one or more of several art-recognized assays, as described in greater detail herein.

As used herein, “L-amino acid” refers to any of the naturally occurring levorotatory alpha-amino acids normally present in proteins or the alkyl esters of those alpha-amino acids. The term “D-amino acid” refers to dextrorotatory alpha-amino acids. Unless specified otherwise, all amino acids referred to herein are L-amino acids.

“Hydrophobic” or “non-polar” are used synonymously herein, and refer to any inter- or intra-molecular interaction not characterized by a dipole.

As used herein, “pharmaceutically acceptable salts” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. Thus, the term “acid addition salt” refers to the corresponding salt derivative of a parent compound that has been prepared by the addition of an acid. The pharmaceutically acceptable salts include the conventional salts or the quaternary ammonium salts of the parent compound formed, for example, from inorganic or organic acids. For example, such conventional salts include, but are not limited to, those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Certain acidic or basic compounds may exist as zwitterions. All forms of the compounds, including free acid, free base, and zwitterions, are contemplated to be within the scope of the present disclosure.

### Compstatin Analogues

Compstatin was first identified as a 27 amino acid peptide and was the first non-host-derived complement inhibitor that was shown to be capable of blocking all three activation pathways (Sahu et al., 1996, J. Immunol., 157: 884-91; U.S. Pat. No. 6,319,897). It is possible to truncate compstatin to a 13 amino acid peptide without loss of activity. However, attempts to further truncate this peptide have led to loss of activity. The sequence of the 13 amino acid truncated (or “core”) compstatin peptide is H-Ile¹-Cys²-Val³-Val⁴-Gln⁵-Asp⁶-Trp⁷-Gly⁸-His⁹-His¹⁰-Arg¹¹-Cys¹²-Thr¹³-NH₂ where Cys² and Cys¹² are disulfide bonded. This cyclic tridecapeptide binds to C3 (and fragments of C3), thereby inhibiting the activation of the downstream complement cascade and preventing the cleavage of native C3 by the C3 convertases. Its inhibitory efficacy was confirmed by a series of studies using experimental models that pointed to its potential as a therapeutic agent (Fiane et al, 1999a, Xenotransplantation, 6: 52-65; Fiane et al, 1999b, Transplant Proc., 31:934-935; Nilsson et al., 1998, Blood, 92: 1661-1667; Ricklin & Lambris, 2008, Adv. Exp. Med. Biol., 632: 273-292; Schmidt et al., 2003, J. Biomed. Mater. Res., A66: 491-499; Soulika et al., 2000, Clin. Immunol., 96: 212-221).

Progressive optimization of the 13 amino acid compstatin peptide led to analogues with improved biological activity (Ricklin & Lambris, 2008, supra; WO 2004/026328; WO 2007/062249, WO 2013/036778, WO 2014/100407). Structure-activity studies identified the cyclic nature of the compstatin peptide and the presence of both a β-turn and hydrophobic cluster as key features of the molecule (Morikis et al., 1998, Protein Sci., 7: 619-627; WO 99/13899; Morikis et al., 2002, J. Biol. Chem., 277:14942-14953; Ricklin & Lambris, 2008, supra). Hydrophobic residues at positions 4 and 7 were found to be of particular importance, and their modification with unnatural amino acids generated an analogue with 264-fold improved activity over the original compstatin peptide (Katragadda et al., 2006, J. Med. Chem., 49: 4616-4622; WO 2007/062249). Further attempts to optimize compstatin for use in the treatment of eye disorders are described in WO 2007/044668.

While previous optimization steps have been based on combinatorial screening studies, solution structures, and computational models (Chiu et al., 2008, Chem. Biol. Drug Des., 72: 249-256; Mulakala et al., 2007, Bioorg. Med. Chem., 15: 1638-1644; Ricklin & Lambris, 2008, supra), the publication of a co-crystal structure of compstatin complexed with the complement fragment C3c (Janssen et al., 2007, J. Biol. Chem., 282: 29241-29247; WO 2008/153963) provides a basis for initiating rational optimization. The crystal structure reveals a shallow binding site at the interface of macroglobulin (MG) domains 4 and 5 of C3c and shows that 9 of the 13 amino acids are directly involved in the binding, either through hydrogen bonds or hydrophobic interactions. As compared to the structure of the compstatin peptide in solution (Morikis et al., 1998, supra), the bound form of compstatin experienced a conformational change, with a shift in the location of the β-turn from residues 5-8 to 8-11 (Janssen et al., 2007, supra; WO 2008/153963).

Ac-Compstatin, an N-terminally acetylated 13 amino acid peptide, binds to C3 and prevents C3 convertase-mediated cleavage. Since its discovery by phage display, modification to the 13 amino acid Ac-Compstatin sequence has been carried out to find analogues with increased biological activity. However, in the core sequence between the two cysteines residues at positions 2 and 12, alanine scanning experiments have previously produced analogues showing only modest improvements in biological activity, with few modifications being tolerated. The modifications include changing the valine at position 4 to tryptophan, or a tryptophan analogue, that leads to an increase in biological activity and changing the histidine at position 9 to alanine or analogs thereof.

Attempts to introduce modifications to the valine residue at position 3, replacing it with glycine, alanine, D-valine or leucine have led to a decrease in biological activity. In contrast to these findings, our studies have shown that a change of valine to isoleucine is well tolerated and provides improvements in biological activity.

Without wishing to be bound by any specific theory, this modification can be combined with the introduction of one or more polar or charged amino acids in the core sequence and may be used as an approach to increase the ability of the compstatin peptides to solubilize. For example, glutamic acid or serine at position 9 may be particularly suitable for combination with isoleucine 3 although they may lead to a decrease in activity when combined with valine 3. These observations correlate with improved binding to C3 as measured by surface plasmon resonance (SPR).

Furthermore, these changes can be readily combined with other modifications in the core sequence of the compstatin analogues and with addition of N- and C-terminal sequences, for example for improving the solubility of the compstatin peptides, e.g., at higher concentrations.

The compstatin analogs described herein typically have greater activity than Ac-compstatin, e.g., at least 10-fold greater activity, at least 20-fold greater activity, at least 30-fold greater activity than Ac-compstatin. In other embodiments, the analogs have at least 40-, 50-, 60-, 70-, 80-, 90-, 100-, 110-, 120-, 130-, 140-, 150-fold or greater activity than Ac-compstatin, as compared utilizing the assays described in the examples.

A compound having Ile at position X3 may have greater activity than an otherwise identical compound having valine at position X3, i.e. the position corresponding to Val3 of compstatin.

The compstatin analogues are capable of binding to C3 and/or C3b, and of inhibiting activation of the complement cascade, particularly downstream of C3, e.g., by inhibiting cleavage of C3 by C3 convertases.

The compstatin analogues are also typically capable of inhibiting complement-driven haemolysis. Complement-driven haemolysis is typically assessed (in a “haemolysis assay”) by contacting serum from a first mammalian species (e.g., human serum) with erythrocytes (red blood cells; RBC) from a second mammalian species (e.g., sheep or any other suitable species), typically in the presence of mammalian immunoglobulin capable of binding to the erythrocytes. Complement in the serum is activated by the cell-bound immunoglobulin, leading to lysis of the erythrocytes, i.e., haemolysis. The immunoglobulin may be from the first species or may be from a third mammalian species as long as it is capable of activating complement from the first species.

In such an assay, a test compound is typically be pre-incubated with the serum before the serum is contacted with the erythrocytes. The erythrocytes may also be pre-incubated with the immunoglobulin before contacting with the serum.

In the examples below, human serum is pre-incubated with a test compound, and sheep erythrocytes are pre-incubated with rabbit anti-serum against sheep erythrocytes, before the serum and erythrocytes are combined.

Thus, the activity of the compstatin analogues may be determined with reference to one or more biological activities selected from (1) binding to C3 protein, (2) binding to C3b protein, (3) inhibiting the cleavage of native C3 by C3 convertases, and (4) inhibiting the activation of the complement system.

A compstatin analogue described herein may bind C3 or C3b with a higher affinity than that of compstatin. For example, they may have a Kd at least 10-fold lower, at least 20-fold lower, or at least 30-fold lower than Ac-compstatin, e.g., at least 40-, 50-, 60-, 70-, 80-, 90-, 100-, 110-, 120-, 130-, 140-, or 150-fold lower than Ac-compstatin. The Kd may be determined, for example, by surface plasmon resonance (SPR), e.g., using an assay as described in Example 3.

A compstatin analogue described herein typically binds C3 or C3b with a greater affinity (i.e., a lower Kd) than that of an otherwise identical compound having valine instead of isoleucine at the position corresponding to Val3 of compstatin.

A compstatin analogue described herein may have a greater ability to inhibit haemolysis than Ac-compstatin. For example, it may inhibit haemolysis with an IC₅₀ at least 10-fold, at least 20-fold, or at least 30-fold lower than Ac-compstatin, e.g., at least 40-, 50-, 60-, 70-, 80-, 90-, 100-, 110-, 120-, 130-, 140-, 150-, 200-, 250-, 300-350-, 400-, 450-, 500-fold lower than Ac-compstatin.

A compstatin analogue having isoleucine at position 3 typically has a greater ability to inhibit haemolysis (i.e., a lower IC₅₀) than an otherwise identical compound having valine instead of isoleucine at the position corresponding to Val3 of compstatin.

Preferably, the in vitro effect of the compounds described herein are assessed by measuring their inhibitory effect on the classical complement pathway in a haemolysis assay, e.g., using the assay described in Example 2.

Compstatin analogues having acylation may have a lower absolute activity than an otherwise identical compound lacking acylation, but have additional benefits including prolonged in vivo half life, which may offset any apparent reduction of absolute activity.

### Synthesis of Compstatin Analogues

Compstatin analogues described herein can be synthesized, for example, by means of solid-phase or liquid-phase peptide synthesis methodology. In this context, reference may be made to WO 98/11125 and, among many others, Fields, G. B. et al., 2002, “Principles and practice of solid-phase peptide synthesis”. In: Synthetic Peptides (2nd Edition), and the Example herein.

A compstatin analogue described herein can be synthesized or produced in a number of ways, including for example, a method comprising (a) synthesizing the compstatin analogues by means of solid-phase or liquid-phase peptide synthesis methodology and recovering the synthesized compstatin analogues thus obtained; or (b) expressing a precursor peptide sequence from a nucleic acid construct that encodes the precursor peptide, recovering the expression product, and modifying the precursor peptide to yield the compstatin analogue.

The precursor peptide may be modified by introduction of one or more non-proteinogenic amino acids, e.g., Aib, Orn, Dap, 1-Me-Trp, 1-Nal, 2-Nal, Sar, γGlu or Dab, or by the introduction of an appropriate terminal groups Y1 and/or Y2.

Expression is typically performed from a nucleic acid encoding the precursor peptide, which may be performed in a cell or a cell-free expression system comprising such a nucleic acid.

For recombinant expression, the nucleic acid fragments encoding the precursor peptide are normally inserted in suitable vectors to form cloning or expression vectors. The vectors can, depending on purpose and type of application, be in the form of plasmids, phages, cosmids, mini-chromosomes, or virus, but also naked DNA, which is only expressed transiently in certain cells is an important vector. Preferred cloning and expression vectors (plasmid vectors) are capable of autonomous replication, thereby enabling high copy-numbers for the purposes of high-level expression or high-level replication for subsequent cloning.

In general, an expression vector can comprise one or more of the following features: a promoter for driving expression of a nucleic acid, optionally a nucleic acid sequence encoding a leader peptide enabling secretion (to the extracellular phase or, where applicable, into the periplasma), a nucleic acid fragment encoding a peptide, and optionally a terminator. Vectors may comprise additional features such as, for example, selectable markers and origins of replication. When operating with expression vectors in producer strains or cell lines it may be preferred that the vector is capable of integrating into the host cell genome. The skilled person is familiar with suitable vectors and is able to design one according to their specific requirements.

The vectors can be used to transform host cells to produce a peptide. Such transformed cells can be cultured cells or cell lines used for propagation of the nucleic acid fragments and vectors, and/or used for recombinant production of the peptides.

Preferred transformed cells are microorganisms such as bacteria [such as the species Escherichia (e.g., E. coli), Bacillus (e.g., Bacillus subtilis), Salmonella, or Mycobacterium (preferably non-pathogenic, e.g., M. bovis BCG), yeasts (e.g., Saccharomyces cerevisiae and Pichia pastoris), and protozoans. Alternatively, the transformed cells may be derived from a multicellular organism, e.g., it may be fungal cell, an insect cell, an algal cell, a plant cell, or an animal cell such as a mammalian cell. For the purposes of cloning and/or optimised expression it is preferred that the transformed cell is capable of replicating the nucleic acid. Cells expressing the nucleic can be used for small-scale or large-scale preparation of the peptides.

When producing the peptide by means of transformed cells, it is convenient, although far from essential, that the expression product is secreted into the culture medium.

### Medical Conditions

In a broad aspect, described herein are compstatin analogues for use as a medicament or for use in therapy.

The compstatin analogues described herein have biological activities of binding to C3 protein and/or inhibiting complement activation. Generally, the compstatin analogues described herein can be used for the treatment or prevention conditions associated with excessive or unwanted activation of the complement system. Complement can be activated through three different pathways: the classical, lectin and alternative pathways. The major activation event that is shared by all three pathways is the proteolytic cleavage of the central protein of the complement system, C3, into its activation products C3a and C3b by C3 convertases. Generation of these fragments leads to the opsonization of pathogenic cells by C3b and iC3b, a process that renders them susceptible to phagocytosis or clearance, and to the activation of immune cells through an interaction with complement receptors (Markiewski & Lambris, 2007, Am. J. Pathol., 171: 715-727). Deposition of C3b on target cells also induces the formation of new convertase complexes and thereby initiates a self-amplification loop. An ensemble of plasma and cell surface-bound proteins carefully regulates complement activation to prevent host cells from self-attack by the complement cascade. The 13 amino acid cyclic tridecapeptide used as a reference point for the design of the compstatin analogues described herein inhibits complement activation by binding to C3 and/or C3b, thereby preventing the cleavage of native C3 by the C3 convertases. The biological activity of the compstatin analogues described herein can be determined in vitro by measuring, for example, their inhibitory effect of the classical complement pathway in a haemolysis assay, for example using a protocol set out in the examples below.

Excessive activation or inappropriate regulation of complement can lead to a number of pathologic conditions, ranging from autoimmune diseases to inflammatory diseases (Holers, 2003, Clin. Immunol., 107: 140-51; Markiewski & Lambris, 2007, supra; Ricklin & Lambris, 2007, Nat. Biotechnol., 25: 1265-75; Sahu et al., 2000, J. Immunol., 165: 2491-9). These conditions include: (1) inhibiting complement activation to facilitate treatment of diseases or conditions including age-related macular degeneration, Stargardt disease, periodontitis, diabetic retinopathy, glaucoma, uveitis, rheumatoid arthritis, spinal cord injury, stroke, multiple sclerosis, Parkinson's disease, Alzheimer's disease, cancer, and respiratory disorders such as asthma, chronic obstructive pulmonary disease (COPD), allergic inflammation, emphysema, bronchitis, bronchiecstasis, cystic fibrosis, tuberculosis, pneumonia, respiratory distress syndrome (RDS—neonatal and adult), rhinitis and sinusitis; bacterial infections such as sepsis, ischemia-reperfusion injury in various tissues, myocardial infarction, anaphylaxis, paroxysmal nocturnal hemoglobinuria, autoimmune hemolytic anemias, psoriasis, hidradentitis suppurativa, myasthenia gravis, systemic lupus erythematosus, CHAPLE syndrome, C3 glomeropathy, IgA nephropathy, atypical hemolytic uremic syndrome, Crohn's disease, ulcerative colitis, antiphospholipid syndrome, or (2) inhibiting complement activation that occurs during cell or solid organ transplantation, or in the use of artificial organs or implants (e.g., by coating or otherwise treating the cells, organs, artificial organs or implants with a peptide of the invention); or (3) inhibiting complement activation that occurs during extracorporeal shunting of physiological fluids (blood, urine) (e.g., by coating the tubing through which the fluids are shunted with a compstatin analogue described herein).

### Pharmaceutical Compositions and Administration

Described herein are composition(s) comprising a compstatin analogue, or a pharmaceutically acceptable salt or solvate thereof, together with a carrier. In one embodiment, the composition is a pharmaceutical composition and the carrier is a pharmaceutically acceptable carrier. Also described herein are pharmaceutical composition(s) comprising a compstatin analogue, or a salt and/or solvate thereof, together with a carrier, excipient or vehicle. Accordingly, the compstatin analogue, or salts or solvates thereof, especially pharmaceutically acceptable salts and/or solvates thereof, may be formulated as compositions or pharmaceutical compositions prepared for storage or administration, and comprise a therapeutically effective amount of a compstatin analogue, or a salt or solvate thereof.

Suitable salts formed with bases include metal salts, such as alkali metal or alkaline earth metal salts.

In one embodiment, a pharmaceutical composition is one wherein the compstatin analogue is in the form of a pharmaceutically acceptable acid addition salt.

As will be apparent to one skilled in the medical art, a “therapeutically effective amount” of a compstatin analogue compound or pharmaceutical composition can vary depending upon, inter alia, the age, weight and/or gender of the subject (patient) to be treated. Other factors that may be of relevance include the physical characteristics of the specific patient under consideration, the patient's diet, the nature of any concurrent medication, the particular compound(s) employed, the particular mode of administration, the desired pharmacological effect(s) and the particular therapeutic indication. Because these factors and their relationship in determining this amount are known in the medical arts, the determination of therapeutically effective dosage levels, the amount necessary to achieve the desired result of treating and/or preventing and/or remedying malabsorption and/or low-grade inflammation described herein, as well as other medical indications disclosed herein, will be within the ambit of the skilled person.

As used herein, the term “therapeutically effective amount” refers to an amount that reduces symptoms of a given condition or pathology, and normalizes physiological responses in an individual with the condition or pathology. Reduction of symptoms or normalization of physiological responses can be determined using methods routine in the art and may vary with a given condition or pathology. In one aspect, a therapeutically effective amount of one or more compstatin analogues, or pharmaceutical compositions thereof, is an amount that restores a measurable physiological parameter to substantially the same value (preferably to within 30%, more preferably to within 20%, and still more preferably to within 10% of the value) of the parameter in an individual without the condition or pathology in question.

In one embodiment, administration of a compound or pharmaceutical composition is commenced at lower dosage levels, with dosage levels being increased until the desired effect of preventing/treating the relevant medical indication is achieved. This defines a therapeutically effective amount. For the compstatin analogues described herein, alone or as part of a pharmaceutical composition, such human doses of the active compstatin analogue may be between about 0.01 pmol/kg and 500 μmol/kg body weight, between about 0.01 pmol/kg and 300 μmol/kg body weight, between 0.01 pmol/kg and 100 μmol/kg body weight, between 0.1 pmol/kg and 50 μmol/kg body weight, between 1 pmol/kg and 10 μmol/kg body weight, between 5 pmol/kg and 5 μmol/kg body weight, between 10 pmol/kg and 1 μmol/kg body weight, between 50 pmol/kg and 0.1 μmol/kg body weight, between 100 pmol/kg and 0.01 μmol/kg body weight, between 0.001 pmol/kg and 0.5 μmol/kg body weight, between 0.05 pmol/kg and 0.1 μmol/kg body weight.

The therapeutic dosing and regimen most appropriate for patient treatment will of course vary with the disease or condition to be treated, and according to the patient's weight and other parameters. Without wishing to be bound by any particular theory, it is expected that doses, in the mg/kg range, and shorter or longer duration or frequency of treatment may produce therapeutically useful results, such as a statistically significant inhibition of the alternative and classical complement pathways. The dosage sizes and dosing regimen most appropriate for human use may be guided by the results obtained by methods known in the art or described herein, and may be confirmed in properly designed clinical trials.

An effective dosage and treatment protocol may be determined by conventional means, starting with a low dose in laboratory animals and then increasing the dosage while monitoring the effects, and systematically varying the dosage regimen as well. Numerous factors may be taken into consideration by a clinician when determining an optimal dosage for a given subject.

For local delivery to the eye, the pharmaceutically acceptable composition(s) may be formulated in isotonic, pH adjusted sterile saline or water, either with or without a preservative such as benzylalkonium chloride. Alternatively, for ophthalmic uses, the pharmaceutically acceptable compositions may be formulated in an ointment such as petrolatum or as eyedrops. Methods of local administration to the eye include, e.g., choroidal injection, transscleral injection or placing a scleral patch, selective arterial catheterization, eyedrops or eye ointments, intraocular administration including transretinal, subconjunctival bulbar, intravitreous injection, suprachoroidal injection, subtenon injection, scleral pocket and scleral cutdown injection, by osmotic pump, etc. The composition(s) can also be alternatively administered intravascularly, such as intravenously (IV) or intraarterially. In choroidal injection and scleral patching, the clinician uses a local approach to the eye after initiation of appropriate anesthesia, including painkillers and ophthalmoplegics. A needle containing the therapeutic compound is directed into the subject's choroid or sclera and inserted under sterile conditions. When the needle is properly positioned the compound is injected into either or both of the choroid or sclera. When using either of these methods, the clinician can choose a sustained release or longer acting formulation. Thus, the procedure can be repeated only every several months or several years, depending on the subject's tolerance of the treatment and response.

The compounds described have particularly advantageous properties as a result of their particular amino acid sequences and/or acylation. They have cysteine residues linked by disulphide bonds at the positions corresponding to positions 2 and 12 of compstatin. It is believed that similar or otherwise identical compounds containing thioether linkages will have similar advantageous properties, and/or will show improvements in stability, such as chemical stability (resistance to degradation) or physical stability (resistance to aggregation).

The following examples are provided to describe the embodiments in greater detail. They are intended to illustrate, not to limit, the scope of the present disclosure.

### EXAMPLES

### Example 1: Synthesis of Compstatin Analogues

### General Peptide Synthesis

List of abbreviations and suppliers

| | Abbre- | | |
|---|---|---|---|
| | viation | Name | Brand/Supplier |
| Resins | | TentaGel ™ SRAM | Rapp Polymere |
| Amino | | Pseudoprolines (e.g., YS, | Jupiter Bioscience Ltd. |
| acids | | FS, FT) | |
| | | Fmoc-L-Aaa-OH | Senn Chemicals AG |
| Coupling | Oxyma | Ethyl cyanoglyoxylate-2- | Chem Impex international |
| reagents | Pure | oxime | |
| | DIC | Diisopropylcarbodiimide | Fluka/Sigma Aldrich Co. |
| | HATU | N-[(dimethylamino)-1H- | ChemPep Inc. |
| | | 1,2,3-triazol[4,5-b]pyridine- | |
| | | 1-ylmethylene]-N- | |
| | | methylmethanaminium | |
| | | hexafluorophosphate N- | |
| | | oxide | |
| | HOBt | Hydroxybenzotriazole | Sigma-Aldrich Co. |
| Solvents | Boc₂O | Di-tert-butyl pyrocarbonate | Advanced ChemTech |
| and | DCM | Dichloromethane | Prolabo (VWR) |
| reagents | DIPEA | Diisopropylethylamine | Fluka/Sigma Aldrich Co. |
| | DMF | N,N-dimethylformamide | Taminco |
| | Et₂O | Diethyl ether | Prolabo (VWR) |
| | EtOH | Ethanol | CCS Healthcare AB |
| | HCOOH | Formic acid (HPLC grade) | Sigma-Aldrich Co. |
| | H₂O | Water, Milli-Q water | Millipore |
| | MeCN | Acetonitrile (HPLC) | Sigma-Aldrich Co. |
| | NMP | N-methylpyrrolidone | Sigma-Aldrich Co. |
| | | Piperidine | Jubliant Life Sciences Ltd. |
| | TFA | Trifluoroacetic acid (HPLC) | Chemicals Raw Materials |
| | | | Ltd. |
| | TIS | Triisopropylsilane | Sigma-Aldrich Co. |
| | DODT | 2,2′- | Sigma-Aldrich Co |
| | | (ethylenedioxy)diethanethiol | |
| Other | MeOH | Methanol | Sigma-Aldrich Co. |
| | | Ascorbic acid | Sigma-Aldrich Co. |
| | I₂ | Iodine | Sigma-Aldrich Co |
| | I₂CH₂ | diiodomethane | Sigma-Aldrich Co |

### Apparatus and Synthetic Strategy

Peptides were synthesized batchwise on a peptide synthesiser, such as a CEM Liberty Peptide Synthesizer or a Symphony X Synthesizer, according to solid phase peptide synthetic procedures using 9-fluorenylmethyl oxycarbonyl (Fmoc) as N-α-amino protecting group and suitable common protection groups for side-chain functionalities.

Polymeric support based resins, such as e.g., TentaGel™, were used. The synthesizer was loaded with resin that prior to usage was swelled in DMF.

### Coupling

### CEM Liberty Peptide Synthesizer

A solution of Fmoc-protected amino acid (4 equiv.) was added to the resin together with a coupling reagent solution (4 equiv.) and a solution of base (8 equiv.). The mixture was either heated by a microwave unit to 70-75° C. and coupled for 5 minutes or coupled with no heat for 60 minutes. During the coupling nitrogen was bubbled through the mixture.

### Symphony X Synthesizer

The coupling solutions were transferred to the reaction vessels in the following order: amino acid (4 equiv.), HATU (4 equiv.) and DIPEA (8 equiv.). The coupling time was 10 min at room temperature (RT) unless otherwise stated. The resin was washed with DMF (5×0.5 min). In case of repeated couplings the coupling time was in all cases 45 min at RT.

### Deprotection

### CEM Liberty Peptide Synthesizer

The Fmoc group was deprotected using piperidine in DMF or other suitable solvents. The deprotection solution was added to the reaction vessel and the mixture was heated for 30 sec. reaching approx. 40° C. The reaction vessel was drained and fresh deprotection solution was added and subsequently heated to 70-75° C. for 3 min. After draining the reaction vessel the resin was washed with DMF or other suitable solvents.

### Symphony X Synthesizer

Fmoc deprotection was performed for 2.5 minutes using 40% piperidine in DMF and repeated using the same conditions. The resin was washed with DMF (5×0.5 min).

### Side Chain Acylation

Fmoc-Lys(Dde)-OH or alternatively another amino acid with an orthogonal side chain protective group was introduced at the position of the acylation (side-chain lipidation). The N-terminus of the linier peptide was protected with Ac or Boc. While the peptide was still attached to the resin, the orthogonal side chain protective group was selectively cleaved using freshly prepared hydrazine hydrate (2-4%) in NMP for 2×15 min. The unprotected lysine side chain was then elongated using standard coupling conditions and Fmoc-deprotections with the desired building block. The lipidation moiety was coupled as the last step.

### Cleavage

The dried peptide resin was treated with TFA and suitable scavengers for approximately 2 hours. The volume of the filtrate was reduced and the crude peptide was precipitated after addition of diethylether. The crude peptide precipitate was washed several times with diethylether and finally dried.

### HPLC Purification of the Crude Peptide

The crude peptide was purified by preparative reverse phase HPLC using a conventional HPLC apparatus, such as a Gilson GX-281 with 331/332 pump combination, for binary gradient application equipped with a column, such as 5×25 cm Gemini NX 5u C18 110A column, and a fraction collector using a flow 20-40 ml/min with a suitable gradient of buffer A (0.1% Fomic acid, aq.) or A (0.1% TFA, aq.) and buffer B (0.1% Formic acid, 90% MeCN, aq.) or B (0.1% TFA, 90% MeCN, aq.). Fractions were analyzed by analytical HPLC and MS and selected fractions were pooled and lyophilized. The final product was characterized by HPLC and MS.

### Formation of Methylene Thioacetal S—CH₂—S

Following purification and lyophilization of the crude linear peptide, the peptide was redissolved in in water and acetonitrile until a clear solution. The concentration of the peptide solution was kept at approx. 5-6 mg/ml depending on the peptides ability to solubilize. The reaction was conducted in a closed container to minimize unwanted air-oxidation. The peptide solution was stirred, while diiodomethane (approx. 20-30 equiv.) and DIPEA (20 equiv.) was added to the peptide solution. After 2-5 hours, the reaction was finished and pH of the reaction mixture was adjusted to pH3 with TFA. The peptide solution was diluted with water before preparative HPLC purification.

### Analytical HPLC

Final purities were determined by analytic HPLC (Agilent 1100/1200 series) equipped with auto sampler, degasser, 20 μl flow cell and Chromeleon software. The HPLC was operated with a flow of 1.2 ml/min at 40° C. using an analytical column, such as Kinetex 2.6 μm XB-C18 100A 100X8, 6 mm column. The compound was detected and quantified at 215 nm. Buffers A (0.1% TFA, aq.) and buffer B (0.1% TFA, 90% MeCN, aq.).

### Mass Spectroscopy

Final MS analysis were determined on a conventional mass spectroscopy, e.g., Waters Xevo G2 TOF, equipped with electrospray ionization with lock-mass calibration and MassLynx software. It was operated in positive mode using direct injection and a cone voltage of 15V (1 TOF), 30 V (2 TOF) or 45 V (3 TOF) as specified on the chromatogram. Precision was 5 ppm with a typical resolution of 15,000-20,000.

### Synthesis of Compound 24

Solid phase peptide synthesis was performed on a Symphony X Synthesizer using standard Fmoc chemistry. TentaGel S RAM (1.3 g; 0.23 mmol/g) was swelled in DMF (3×10 ml) prior to use and the Fmoc-group was deprotected according to the procedure described above.

### Coupling

Suitable protected Fmoc-amino acids according to the sequence were coupled as described above using HATU as coupling reagent. All couplings were performed at R.T. The lysine used for the incorporation of the branched moiety was incorporated as Fmoc-Lys(Dde)-OH for orthogonal coupling.

### Deprotection

Fmoc deprotection was performed according to the procedure described above.

### Side Chain Acylation

While the peptide was still attached to the resin, the orthogonal side-chain protective group (Dde) was selectively cleaved using freshly prepared hydrazine hydrate (2-4%) in NMP for 2×15 min. The unprotected lysine side-chain was doubled coupled with Fmoc-Glu-OtBu followed by single couplings with Fmoc-Gly-OH, Fmoc-Glu-OtBu, and lastly the fatty acid moiety 17-carboxy-heptadecanoic acid mono tert-butyl ester using standard coupling conditions.

Cleavage of the Peptide from the Solid Support

The peptide-resin was washed with EtOH (3×15 ml) and Et2O (3×150 ml) and dried to constant weight at room temperature (r.t.). The peptide was cleaved from the resin by treatment with TFA/TIS/Water (95/2.5/2.5; 40 ml, 2 h; r.t.). The volume of the filtrate was reduced and the crude peptide was precipitated after addition of diethylether. The crude peptide precipitate was washed several times with diethylether and finally dried to constant weight at room temperature yield 913 mg crude peptide product (purity ˜37%).

### HPLC Purification of the Crude Linear Peptide

The crude peptide was purified by preparative reverse phase HPLC using a Gilson GX-281 with 331/332 pump combination for binary gradient application equipped with a 5×25 cm Gemini NX 5u C18 110A, column and a fraction collector and run at 35 ml/min with a gradient of buffer A (0.1% TFA, aq.) and buffer B (0.1% TFA, 90% MeCN, aq.) gradient from 44% B to 69% B in 47 min. Fractions were analyzed by analytical HPLC and MS and relevant fractions were pooled and lyophilized to yield 167 mg, with a purity of 90% as characterized by HPLC and MS as described above. Calculated monoisotopic MW=3665.67 found 3665.66.

### Formation of the Methylene Thioacetal Linkage on the Crude Linear Peptide

The 167 mg purified linear peptide was dissolved in 40 ml water:acetonitrile (1:1). The concentration of the peptide solution was kept at approx. 6 mg/ml depending on the peptides ability to solubilize. The reaction was conducted in a closed container to minimize unwanted air-oxidation. The peptide solution was stirred, while diiodomethane (approx. 20-30 equiv.) and DIPEA (20 equiv.) was added to the peptide solution. The reaction was followed by analytic HPLC but after 3 hours, the reaction was finished and pH of the reaction mixture was adjusted to pH 3 with TFA. The peptide solution was diluted with water before preparative HPLC purification.

### HPLC Purification of the Oxidized Peptide

The crude peptide was purified by preparative reverse phase HPLC using a Gilson GX-281 with 331/332 pump combination for binary gradient application equipped with a 5×25 cm Gemini NX 5u C18 110A, column and a fraction collector and run at 35 ml/min with a gradient of buffer A (0.1% TFA, aq.) and buffer B (0.1% TFA, 90% MeCN, aq.) gradient from 30% B to 60% B in 47 min. Fractions were analysed by analytical HPLC and MS and relevant fractions were pooled and lyophilized to yield 99.6 mg, with a purity of 90% as characterized by HPLC and MS as described above. Calculated monoisotopic MW=3677.69 found 3677.60.

TABLE 1
Synthesized compounds

| Compound | |
|---|---|
| No. | Sequence |
| 1 | Ac-SEF[C(1)]I[[1-Me-Trp]QDWGEHR[C(1)]TGAES-[NH₂] |
| 2 | Ac-SEF[C(1)]I[1-Me-Trp]QDWGEHR[C(1)][Sar]EGE[Peg3][Peg3]-K([17- |
| | Carboxy-heptadecanoyl][yGlu]G[yGlu])-NH₂ |
| 3 | Ac-EGSAY[C(1)]I[1-Me-Trp]QDWGEHR[C(1)][Sar]EK[yGlu]-K([17-Carboxy- |
| | heptadecanoyl][yGlu][Peg3][Peg3])-NH₂ |
| 4 | Ac-SEF[C(1)]I[1-Me-Trp]QDWGEHR[C(1)][Sar]EGA-K([17-Carboxy- |
| | heptadecanoyl]-[yGlu]G[Peg3][yGlu][Peg3]-NH₂ |
| 5 | Ac-SEF[C(1)]I[1-Me-Trp]QDWGEHR[C(1)]TEGE[Peg3]ES-K([17-Carboxy- |
| | heptadecanoyl]-[yGlu]-NH₂ |
| 6 | Ac-GEF[C(1)]I[1-Me-Trp]QDWGEHR[C(1)][Sar]EAE[Peg3][Peg3]-K([17- |
| | Carboxy-heptadecanoyl][yGlu]G[yGlu])-NH₂ |
| 7 | Ac-SEF[C(1)]I[[1-Me-Trp]QDW[Sar]EHR[C(1)][Sar]E[Peg3][Peg3]-K([17- |
| | Carboxy-heptadecanoyl][yGlu]G[yGlu])-NH₂ |
| 8 | Ac-SEF[C(1)]I[1-Me-Trp]QDW[Sar]EHR[C(1)][Sar]E[Peg3][Peg3]-K([17- |
| | Carboxy-heptadecanoyl]-[yGlu]G[yGlu]-NH₂ |
| 9 | Ac-SEF[C(1)]I[[1-Me-Trp]QDW[Sar]EHR[C(1)]TEGE[Peg3][Peg3]-K([17- |
| | Carboxy-heptadecanoyl][yGlu]G[yGlu])-NH₂ |
| 10 | Ac-SEF[C(1)]I[1-Me-Trp]QDW[Sar]EHR[C(1)]TE[Peg3][Peg3]-K([17- |
| | Carboxy-heptadecanoyl][yGlu]G[yGlu])-NH₂ |
| 11 | Ac-SEF[C(1)]I[[1-Me-Trp]QDW[Sar]EHR[C(1)]TEGE[Peg3]ES-K([17- |
| | Carboxy-heptadecanoyl][yGlu])-NH₂ |
| 12 | Ac-EF[C(1)]I[1-Me-Trp]QDW[Sar]EHR[C(1)]TEA[Peg3][Peg3]-K([17- |
| | Carboxy-heptadecanoyl][yGlu]G[yGlu])-NH₂ |
| 13 | Ac-SEF[C(1)]I[1-Me-Trp]QDW[SarJAHR[C(1)]TEGE[Peg3]ES-K([17- |
| | Carboxy-heptadecanoyl][yGlu])-NH₂ |
| 14 | Ac-SEF[C(1)]I[1-Me-Trp]QDWGEHR[C(1)]TGAES[Peg3][Peg3]-K([17- |
| | Carboxy-heptadecanoyl][yGlu]G[yGlu])-NH₂ |
| 15 | Ac-{d}YI[C(1)]I[1-Me-Trp]QDW[Sar]EHR[C(1)]TEGE[Peg3]ES-K([17- |
| | Carboxy-heptadecanoyl][yGlu])-NH₂ |
| 16 | Ac-SEF[C(1)]IWQDW[Sar]EHR[C(1)]TEGE[Peg3]ES-K([17-Carboxy- |
| | heptadecanoyl][yGlu])-NH₂ |
| 17 | Ac-SEF[C(1)]IYQDW[Sar]EHR[C(1)]TEGE[Peg3]ES-K([17-Carboxy- |
| | heptadecanoy!][yGlu])-NH₂ |
| 18 | Ac-SEY[C(1)]I[1-Me-Trp]QDW[Sar]EHR[C(1)]TEGE[Peg3]ES-K([17- |
| | Carboxy-heptadecanoyl][yGlu])-NH₂ |
| 19 | [15-Carboxy-pentadecanoyl]-EGSEF[C(1)]I[1-Me- |
| | TrpJQDW[Sar]EHR[C(1)]TEGE-[NH₂] |
| 20 | Ac-EGSEF[C(1)]I[1-Me-Trp]QDW[Sar]EHR[C(1)]TEGE[Peg3]ES-K([17- |
| | Carboxy-heptadecanoyl][yGlu])-NH₂ |
| 21 | Ac-KSGEF[C(1)]I[1-Me-Trp]QDW[Sar]EHR[C(1)]TEGE[Peg3][Peg3]-K([17- |
| | Carboxy-heptadecanoyl][yGlu]G[yGlu])-NH₂ |
| 22 | Ac-EQEVF[C(1)]I[1-Me-Trp]QDW[Sar]EHR[C(1)]TEGE[Peg3][Peg3]-K([17- |
| | Carboxy-heptadecanoyl][yGlu]G[yGlu])-NH₂ |
| 23 | Ac-ESQVF[C(1)]I[1-Me-Trp]QDW[Sar]EHR[C(1)]TEGE[Peg3][Peg3]-K([17- |
| | Carboxy-heptadecanoyI][yGlu]G[yGlu])-NH₂ |
| 24 | Ac-ESEQVF[C(1)]I[1-Me-Trp]QDW[Sar]EHR[C(1)]TEGE[Peg3][Peg3]-K([17- |
| | Carboxy-heptadecanoyl][yGlu]G[yGlu])-NH₂ |
| 25 | Ac-SEQAF[C(1)]I[1-Me-Trp]QDW[Sar]EHR[C(1)]TEGE[Peg3][Peg3]-K([17- |
| | Carboxy-heptadecanoyl][yGlu]G[yGlu])-NH₂ |
| 26 | Ac-SKQEF[C(1)]I[1-Me-Trp]QDW[Sar]EHR[C(1)]TEGE[Peg3][Peg3]-K([17- |
| | Carboxy-heptadecanoyl][yGlu]G[yGlu])-NH₂ |
| 27 | Ac-EGESGF[C(1)]I[1-Me-Trp]QDW[Sar]EHR[C(1)]TEGE[Peg3][Peg3]-K([17- |
| | Carboxy-heptadecanoyl][yGlu]G[yGlu])-NH₂ |
| 28 | Ac-GQSAF[C(1)]I[1-Me-Trp]QDW[Sar]EHR[C(1)]TEGE[Peg3][Peg3]-K([17- |
| | Carboxy-heptadecanoyl][yGlu]G[yGlu])-NH₂ |
| 29 | H-ESGVF[C(1)]I[1-Me-Trp]QDW[Sar]EHR[C(1)]TEGE[Peg3][Peg3]-K([17- |
| | Carboxy-heptadecanoyl][yGlu]G[yGlu])-NH₂ |
| 30 | Ac-YEQAF[C(1)]I[1-Me-Trp]QDW[Sar]EHR[C(1)]TEGE[Peg3]ES-K([17- |
| | Carboxy-heptadecanoyl][yGlu]G[yGlu])-NH₂ |
| 31 | Ac-SEFC(2)I[1-Me-Trp]QDWGEHRC(2)TGAES-NH₂ |
| 32 | IC(*)VVQDWGHHRC(*)T |
| 33 | H-{d}YIC(*)V[1-Me-Trp]QDW[SarJAHRC(*)[N-Me-Ile]-NH₂ |
| 34 | IC(*)VVQDWGHHRC(*)TAGHMANLTSHASAI |

*Cp40-decribed by Qu et al., Immunobiology 2013, 281(4): 496-505 (also referred to in that paper as ″peptide 14).

The side chains of cysteine residues designated “C(1)” are linked by a methylene thioacetal linkage; that is the sulphur atoms of each cysteine residue are bridged via a methylene group, i.e. —S—CH₂—S—.

The side chains of cysteine residues designated “C(2)” are linked by an ethylene linkage; that is the sulphur atoms of each cysteine residue are bridged via an ethylene group, i.e. —S—CH₂—CH₂—S—.

The side chains of cysteine residues designated “C(*)” are linked by a disulphide bond.

### Example 2: In Vitro Haemolysis Assay

### Method

The in vitro effect of some of the compounds described herein was assessed by measuring their inhibitory effect of the classical complement pathway in a haemolysis assay.

Briefly, compounds described herein and reference compounds were dissolved in DMSO and diluted in Tris/Casein Assay Buffer (10 mM Tris, 145 mM NaCl, 0.5 mM MgCl₂, 0.15 mM CaCl₂, and 0.1% W/V casein, adjusted to pH 7.4) as 9-point serial dilutions in a 96-well plate. Sensitized sheep red blood cells (RBC) coated with rabbit anti-sheep erythrocyte antiserum (Complement Technology, Inc., TX, USA) were washed in Tris/asein Assay Buffer. 50 μL from each well of diluted compound was added to a 96-well plate containing 50 μL diluted human serum (Complement Technology, Inc., TX, USA) and incubated for 15 minutes at room temperature. The serum dilution factor was optimized for every serum batch to obtain 70-90% of maximal haemolysis using the protocol. Then 50 μL sensitized sheep RBCs were added to all wells (107 per well).

After 30 minutes of incubation at 37° C. with gentle agitation, the reaction was stopped by addition of 50 μL Tris STOP Buffer per well (10 mM EDTA, 10 mM Tris, 145 mM NaCl adjusted to pH 7.4). The RBCs were then removed by centrifugation and the resulting supernatant measured for hemolysis by absorbance at 405 nm.

The response was normalized relative to a positive and negative control (vehicle) to calculate the IC50 (nM) from the concentration response curve using the 4-parameter logistic (4PL) nonlinear model for curve fitting (Table 2). All values are based on n=>2 independent determinations.

TABLE 2
in vitro analysis of inhibition of hemolysis

| Comp No | IC50 [nM] |
|---|---|
| 1 | <100 |
| 2 | <500 |
| 3 | <500 |
| 4 | <500 |
| 5 | <500 |
| 6 | <500 |
| 7 | <500 |
| 8 | <500 |
| 9 | <250 |
| 10 | <250 |
| 11 | <250 |
| 12 | <100 |
| 13 | <250 |
| 14 | <250 |
| 15 | <250 |
| 16 | <1000 |
| 17 | <500 |
| 18 | <250 |
| 19 | <250 |
| 20 | <250 |
| 21 | <100 |
| 22 | <100 |
| 23 | <100 |
| 24 | <100 |
| 25 | <100 |
| 26 | <100 |
| 27 | <100 |
| 28 | <100 |
| 29 | <100 |
| 30 | <100 |
| 31 | <250 |
| Cp40 | <100 |

### Example 3: Affinity Measurements by Surface Plasmon Resonance (SPR)

### Method

Surface plasmon resonance (SPR) was used to characterize peptides with respect to their binding affinity (Kd) for C3. Human C3 (Complement tech cat #A113c) was immobilised on the active flow cell of a CM5 sensor chip (GE Healthcare) using standard amine coupling to a density of approximately 3000 resonance units (RU) in a buffer consisting of 10 mM phosphate pH 7.4, 150 mM NaCl, 0.05% Tween20.

For interaction experiments, a multi-cycle experiment approach was used and performed using a BiacoreX100™ instrument (GE Healthcare) at 25° C. Peptides were injected in increasing concentration series (5 different concentrations and buffer reference) for 180 seconds at a flow rate of 30 μL/min in a buffer consisting of 50 mM Tris buffer at pH 7.4, with 150 mM NaCl and 0.05% Tween20. This was followed by a dissociation period for 10 min. The C3 surface was regenerated between runs by two consecutive injections of 3 M MgCl₂ each for 45 seconds.

Sensorgrams were double-referenced (reference surface, blanks) prior to analysis of the kinetic profiles by globally fitting data to a 1:1 Langmuir binding model to obtain association and dissociation rates for calculation of the equilibrium dissociation constant Kd (Table 3). Each peptide was tested at in at least 2 independent experiments.

TABLE 3
Compstatin analogues binding affinities for C3 as determined
by a surface plasmon resonance assay with immobilized C3.

| Compound | Kd [nM] |
|---|---|
| 1 | 25 |
| 2 | 54 |
| 3 | NT |
| 4 | NT |
| 5 | NT |
| 6 | NT |
| 7 | 21 |
| 8 | NT |
| 9 | 17 |
| 10 | 19 |
| 11 | NT |
| 12 | 12 |
| 13 | NT |
| 14 | NT |
| 15 | 16 |
| 16 | NT |
| 17 | NT |
| 18 | NT |
| 19 | 19 |
| 20 | 19 |
| 21 | NT |
| 22 | NT |
| 23 | 14 |
| 24 | 13 |
| 25 | 9.1 |
| 26 | 11 |
| 27 | 17 |
| 28 | 15 |
| 29 | 15 |
| 30 | NT |
| Cp40 | 0.5 |

NT = Not Tested

All values are based on n=>2 independent determinations

### Example 4: Profiling of Test Compounds in Non-Human Primates (NHP)

Healthy male Cynomolgus monkeys (Macaca fascicularis) received single subcutaneous administrations of each test substance. Compounds were formulated in 20 mM phosphate adjusted with NaOH to pH 7.5 and mannitol for isotonicity and dosed at 1840 nmol/kg. Blood was collected from a femoral vein from each animal at the following times: pre-dose, 1, 2, 4, 8, 24, 48, 72, 96 and 120 h (10 sampling times). Blood was collected into serum separation tubes and allowed to clot at room temperature. The tubes were centrifuged and resulting serum was aliquoted and snap-frozen over dry-ice and stored at nominally −80° C. until analysis. All NHP studies were performed in accordance with animal welfare laws and regulations, including approval of the study by a local ethical review process.

Serum isolated from non-human primates at specific time points after dosing were analyzed for alternative pathway complement activity using the Complement system Alternative Pathway WIESLAB® from Svar Life Science (previously Euro diagnostic AB, Sweden) following the manufacturer's protocol. Briefly, serum samples or controls were diluted in buffer and incubated in microtitre strips coated with specific activators of the alternative pathway. The wells were washed and formed C5b-9 was detected using included colorimetric reagents. Absorbance at 405 nm was measured. The percent activity of the alternative complement pathway was calculated for each animal and timepoint relative to the pre-dose activity (0 hours) of the individual animal with subtraction of the negative control. This reflects the pharmacological activity of the compounds.

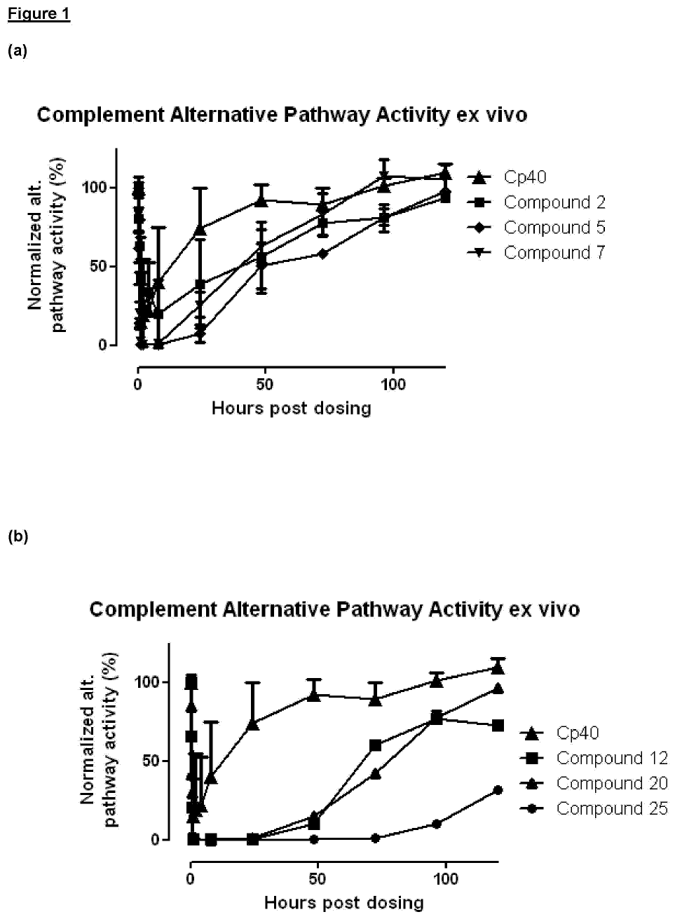

The results from the Alternative Pathway WIESLAB® kit are shown in FIGS. 1a and 1b.

1. A compstatin analogue represented by Formula I:
Y1-R1-X1-C-X3-X4-Q-X6-W-X8-X9-H-X11-C-X13-R2-Y2 (1)
wherein:
Y1 is hydrogen, acetyl or a lipophilic group ϕ;
X1 is I, Y, F or Sar;
X3 is I or V;
X4 is W, F, V, Y, 1-Me-Trp, D-Trp, N-Me-Trp, 1-For-Trp, 1-Nal, 2-Nal, 5MeTrp, Bpa or 2Igl;
X6 is E or D;
X8 is G or Sar;
X9 is H, A, E, D, K, R or S;
X11 is R, K or S;
X13 is T, S, E, F, H, K, Sar, G, I, D, N-Me-Ile or N-Me-Thr;
Y2 is NH₂, OH or a lipophilic group ϕ;
R1 is absent or is a sequence of 1 to 6 amino acid residues selected from A, E, G, L, K, F, P, S, T, W, R, Q, Y, V or Sar, or a corresponding D form thereof; and
R2 is absent or is a sequence of 1 to 6 amino acid residues selected from A, E, G, L, K, F, P, S, T, W, R, V, 8-Amino-3,6-dioxaoctanoyl (Peg3), Sar, γGlu or a corresponding D form thereof;
wherein the compstatin analogue optionally comprises at least one lipophilic group ϕ covalently linked to the side chain of one or more amino acid residues; and
wherein said compstatin analogue has a C₁₋₃alkylene bridge between the sulphur atoms of the cysteine residues at positions 2 and 12;
or a pharmaceutically acceptable salt or solvate thereof.

2. A compstatin analogue represented by Formula II:
Y1-R1-X1-C-I-X4-Q-X6-W-X8-X9-H-X11-C-X13-R2-Y2 (II)
wherein:
Y1 is hydrogen, acetyl or a lipophilic group ϕ;
X1 is I, Y, F or Sar;
X4 is W, F, V, Y, 1-Me-Trp, D-Trp, N-Me-Trp, 1-For-Trp, 1-Nal, 2-Nal, 5MeTrp, Bpa or 2Igl;
X6 is E or D;
X8 is G or Sar;
X9 is H, A, E, D, K, R or S;
X11 is R, K or S;
X13 is T, S, E, F, H, K, Sar, G, I, D, N-Me-Ile or N-Me-Thr;
Y2 is NH₂, OH or a lipophilic group ϕ;
R1 is absent or is a sequence of 1 to 6 amino acid residues selected from A, E, G, L, K, F, P, S, T, W, R, Q, Y, V or Sar, or a corresponding D form thereof; and
R2 is absent or is a sequence of 1 to 6 amino acid residues selected from A, E, G, L, K, F, P, S, T, W, R, V, 8-Amino-3,6-dioxaoctanoyl (Peg3), Sar, γGlu or a corresponding D form thereof;
wherein the compstatin analogue optionally comprises at least one lipophilic group ϕ covalently linked to the side chain of one or more amino acid residues; and
wherein said compstatin analogue has a C₁₋₃alkylene bridge between the sulphur atoms of the cysteine residues at positions 2 and 12;
or a pharmaceutically acceptable salt or solvate thereof.

3. A compstatin analogue or pharmaceutically acceptable salt or solvate according to claim 1 or claim 2 wherein:
X1 is I, Y or F;
X4 is W, Y, 1-Me-Trp, 1-Nal, 2-Nal;
X6 is E or D;
X8 is G or Sar;
X9 is A or E;
X11 is R or K; and
X13 is T, S, E, F, H, K, Sar, G, I, D, N-Me-Ile or N-Me-Thr.

4. A compstatin analogue represented by Formula III:
Y1-R1-F-C-1-1-Me-Trp-Q-X6-W-X8-E-H-R-C-X13-R2-Y2 (III)
wherein:
Y1 is hydrogen, acetyl or a lipophilic group ϕ;
X6 is E or D;
X8 is G or Sar;
X13 is T or Sar;
Y2 is NH₂, OH or a lipophilic group ϕ;
R1 is absent or is a sequence of 1 to 6 amino acid residues selected from A, E, G, L, K, F, P, S, T, W, R, Q, Y, V or Sar, or a corresponding D form thereof; and
R2 is absent or is a sequence of 1 to 6 amino acid residues selected from A, E, G, L, K, F, P, S, T, W, R, V, 8-Amino-3,6-dioxaoctanoyl (Peg3), Sar, γGlu or a corresponding D form thereof;
wherein the compstatin analogue optionally comprises at least one lipophilic group ϕ covalently linked to the side chain of one or more amino acid residues; and
wherein said compstatin analogues has a C₁₋₃alkylene bridge between the sulphur atoms of the cysteine residues at positions 2 and 12;
or a pharmaceutically acceptable salt or solvate thereof.

5. A compstatin analogue or pharmaceutically acceptable salt or solvate according to any one of the preceding claims wherein R1 is selected from
SE, EGSA, GE, E, {d}Y, EGSE, KSGE, EQEV, ESQV, ESEQV, SEQA, SKQE, EGESG, GQSA, ESGV and YEQA.

6. A compstatin analogue according to any one of the preceding claims wherein R2 is selected from
GAES,
EGE[Peg3][Peg3]-K*,
EK[γGlu]-K*,
EGA-K*
EGE[Peg3]ES-K*,
EAE[Peg3][Peg3]-K*,
E[Peg3][Peg3]-K*,
EA[Peg3][Peg3]-K*, and
GAES[Peg3][Peg3]-K*.

7. A compstatin analogue or pharmaceutically acceptable salt or solvate according to any one of the preceding claims wherein the peptide backbone is selected from:

| | |
|---|---|
| 1 | SEF[C(1)]I[1-Me-Trp]QDWGEHR[C(1)]TGAES |
| 2 | SEF[C(1)]I[1-Me-Trp]QDWGEHR[C(1)][Sar]EGE[Peg3][Peg3]-[K*] |
| 3 | EGSAY[C(1)]I[1-Me-Trp]QDWGEHR[C(1)][Sar]EK[yGlu]-[K*] |
| 4 | Ac-SEF[C(1)]I[1-Me-Trp]QDWGEHR[C(1)][Sar]EGA-[K*] |
| 5 | SEF[C(1)]I[1-Me-Trp]QDWGEHR[C(1)]TEGE[Peg3]ES-[K*] |
| 6 | GEF[C(1)]I[1-Me-Trp]QDWGEHR[C(1)][Sar]EAE[Peg3][Peg3]-[K*] |
| 7 | SEF[C(1)]I[1-Me-Trp]QDW[Sar]EHR[C(1)][Sar]E[Peg3][Peg3]-[K*] |
| 8 | SEF[C(1)]I[1-Me-Trp]QDW[Sar]EHR[C(1)][Sar]E[Peg3][Peg3]-[K*] |
| 9 | SEF[C(1)]I[1-Me-Trp]QDW[Sar]EHR[C(1)]TEGE[Peg3][Peg3]-[K*] |
| 10 | SEF[C(1)]I[1-Me-Trp]QDW[Sar]EHR[C(1)]TE[Peg3][Peg3]-[K*] |
| 11 | SEF[C(1)]I[1-Me-Trp]QDW[Sar]EHR[C(1)]TEGE[Peg3]ES-[K*] |
| 12 | EF[C(1)]I[1-Me-Trp]QDW[Sar]EHR[C(1)]TEA[Peg3][Peg3]-[K*] |
| 13 | SEF[C(1)]I[1-Me-Trp]QDW[SarJAHR[C(1)]TEGE[Peg3]ES-[K*] |
| 14 | SEF[C(1)]I[1-Me-Trp]QDWGEHR[C(1)]TGAES[Peg3][Peg3]-[K*] |
| 15 | {d}YI[C(1)]I[1-Me-Trp]QDW[Sar]EHR[C(1)]TEGE[Peg3]ES-[K*] |
| 16 | SEF[C(1)]IWQDW[Sar]EHR[C(1)]TEGE[Peg3]ES-[K*] |
| 17 | SEF[C(1)]IYQDW[Sar]EHR[C(1)]TEGE[Peg3]ES-[K*] |
| 18 | SEY[C(1)]I[1-Me-Trp]QDW[Sar]EHR[C(1)]TEGE[Peg3]ES-[K*] |
| 19 | EGSEF[C(1)]I[1-Me-Trp]QDW[Sar]EHR[C(1)]TEGE |
| 20 | EGSEF[C(1)]I[1-Me-Trp]QDW[Sar]EHR[C(1)]TEGE[Peg3]ES-[K*] |
| 21 | KSGEF[C(1)]I[1-Me-Trp]QDW[Sar]EHR[C(1)]TEGE[Peg3][Peg3]-[K*] |
| 22 | EQEVF[C(1)]I[1-Me-Trp]QDW[Sar]EHR[C(1)]TEGE[Peg3][Peg3]-[K*] |
| 23 | ESQVF[C(1)]I[1-Me-Trp]QDW[Sar]EHR[C(1)]TEGE[Peg3][Peg3]-[K*] |
| 24 | ESEQVF[C(1)]I[1-Me-Trp]QDW[Sar]EHR[C(1)]TEGE[Peg3][Peg3]-[K*] |
| 25 | SEQAF[C(1)]I[1-Me-Trp]QDW[Sar]EHR[C(1)]TEGE[Peg3][Peg3]-[K*] |
| 26 | SKQEF[C(1)]I[1-Me-Trp]QDW[Sar]EHR[C(1)]TEGE[Peg3][Peg3]-[K*] |
| 27 | EGESGF[C(1)]I[1-Me-Trp]QDW[Sar]EHR[C(1)]TEGE[Peg3][Peg3]-[K*] |
| 28 | GQSAF[C(1)]I[1-Me-Trp]QDW[Sar]EHR[C(1)]TEGE[Peg3][Peg3]-[K*] |
| 29 | ESGVF[C(1)]I[1-Me-Trp]QDW[Sar]EHR[C(1)]TEGE[Peg3][Peg3]-[K*] |
| 30 | YEQAF[C(1)]I[1-Me-Trp]QDW[Sar]EHR[C(1)]TEGE[Peg3]ES-[K*] |
| 31 | SEFC(2)I[1-Me-Trp]QDWGEHRC(2)TGAES |

where [C(1)] indicates pairs of cysteine residues having a methylene bridging group between the sulphur atoms of their side chains their side chains, where [C(2)] indicates pairs of cysteine residues having an ethylene bridging group between the sulphur atoms of their side chains, and where * indicates that the amino acid residue bears a lipophilic group ϕ covalently linked to its side chain.

8. A compstatin analogue or pharmaceutically acceptable salt or solvate according to claim 7 wherein the peptide backbone is selected from:

| | |
|---|---|
| 1 | SEF[C(1)]I[1-Me-Trp]QDWGEHR[C(1)]TGAES |
| 2 | SEF[C(1)]I[1-Me-Trp]QDWGEHR[C(1)][Sar]EGE[Peg3][Peg3]-K([17- |
| | Carboxy-heptadecanoyl][yGlu]G[yGlu]) |
| 3 | EGSAY[C(1)]I[1-Me-Trp]QDWGEHR[C(1)][Sar]EK[yGlu]-K([17- |
| | Carboxy-heptadecanoyl][yGlu][Peg3][Peg3]) |
| 4 | Ac-SEF[C(1)]I[1-Me-Trp]QDWGEHR[C(1)][Sar]EGA-K([17-Carboxy- |
| | heptadecanoyl]-[yGlu]G[Peg3][yGlu][Peg3]) |
| 5 | SEF[C(1)]I[1-Me-Trp]QDWGEHR[C(1)]TEGE[Peg3]ES-K([17- |
| | Carboxy-heptadecanoyl]-[yGlu]) |
| 6 | GEF[C(1)]I[1-Me-Trp]QDWGEHR[C(1)][Sar]EAE[Peg3][Peg3]-K([17- |
| | Carboxy-heptadecanoyl][yGlu]G[yGlu]) |
| 7 | SEF[C(1)]I[1-Me-Trp]QDW[Sar]EHR[C(1)][Sar]E[Peg3][Peg3]-K([17- |
| | Carboxy-heptadecanoyl][yGlu]G[yGlu]) |
| 8 | SEF[C(1)]I[1-Me-Trp]QDW[Sar]EHR[C(1)][Sar]E[Peg3][Peg3]-K([17- |
| | Carboxy-heptadecanoyl]-[yGlu]G[yGlu]) |
| 9 | SEF[C(1)]I[1-Me-Trp]QDW[Sar]EHR[C(1)]TEGE[Peg3][Peg3]-K([17- |
| | Carboxy-heptadecanoyl][yGlu]G[yGlu]) |
| 10 | SEF[C(1)]I[1-Me-Trp]QDW[Sar]EHR[C(1)]TE[Peg3][Peg3]-K([17- |
| | Carboxy-heptadecanoyl][yGlu]G[yGlu]) |
| 11 | SEF[C(1)]I[1-Me-Trp]QDW[Sar]EHR[C(1)]TEGE[Peg3]ES-K([17- |
| | Carboxy-heptadecanoyl][yGlu]) |
| 12 | EF[C(1)]I[1-Me-Trp]QDW[Sar]EHR[C(1)]TEA[Peg3][Peg3]-K([17- |
| | Carboxy-heptadecanoyl][yGlu]G[yGlu]) |
| 13 | SEF[C(1)]I[1-Me-Trp]QDW[SarJAHR[C(1)]TEGE[Peg3]ES-K([17- |
| | Carboxy-heptadecanoyl][yGlu]) |
| 14 | SEF[C(1)]I[1-Me-Trp]QDWGEHR[C(1)]TGAES[Peg3][Peg3]-K([17- |
| | Carboxy-heptadecanoyl][yGlu]G[yGlu]) |
| 15 | {d}YI[C(1)]I[1-Me-Trp]QDW[Sar]EHR[C(1)]TEGE[Peg3]ES-K([17- |
| | Carboxy-heptadecanoyl][yGlu]) |
| 16 | SEF[C(1)]IWQDW[Sar]EHR[C(1)]TEGE[Peg3]ES-K([17-Carboxy- |
| | heptadecanoyl][yGlu]) |
| 17 | SEF[C(1)]IYQDW[Sar]EHR[C(1)]TEGE[Peg3]ES-K([17-Carboxy- |
| | heptadecanoyl][yGlu]) |
| 18 | SEY[C(1)]I[1-Me-Trp]QDW[Sar]EHR[C(1)]TEGE[Peg3]ES-K([17- |
| | Carboxy-heptadecanoyl][yGlu]) |
| 19 | EGSEF[C(1)]I[1-Me-Trp]QDW[Sar]EHR[C(1)]TEGE |
| 20 | EGSEF[C(1)]I[1-Me-Trp]QDW[Sar]EHR[C(1)]TEGE[Peg3]ES-K([17- |
| | Carboxy-heptadecanoyl][yGlu]) |
| 21 | KSGEF[C(1)]I[1-Me-Trp]QDW[Sar]EHR[C(1)]TEGE[Peg3][Peg3]- |
| | K([17-Carboxy-heptadecanoyl][yGlu]G[yGlu]) |
| 22 | EQEVF[C(1)]I[1-Me-Trp]QDW[Sar]EHR[C(1)]TEGE[Peg3][Peg3]- |
| | K([17-Carboxy-heptadecanoyl][yGlu]G[yGlu]) |
| 23 | ESQVF[C(1)]I[1-Me-Trp]QDW[Sar]EHR[C(1)]TEGE[Peg3][Peg3]- |
| | K([17-Carboxy-heptadecanoyl][yGlu]G[yGlu]) |
| 24 | ESEQVF[C(1)]I[1-Me-Trp]QDW[Sar]EHR[C(1)]TEGE[Peg3][Peg3]- |
| | K([17-Carboxy-heptadecanoyl][yGlu]G[yGlu]) |
| 25 | SEQAF[C(1)]I[1-Me-Trp]QDW[Sar]EHR[C(1)]TEGE[Peg3][Peg3]- |
| | K([17-Carboxy-heptadecanoyI][yGlu]G[yGlu]) |
| 26 | SKQEF[C(1)]I[1-Me-Trp]QDW[Sar]EHR[C(1)]TEGE[Peg3][Peg3]- |
| | K([17-Carboxy-heptadecanoyl][yGlu]G[yGlu]) |
| 27 | EGESGF[C(1)]I[1-Me-Trp]QDW[Sar]EHR[C(1)]TEGE[Peg3][Peg3]- |
| | K([17-Carboxy-heptadecanoyI][yGlu]G[yGlu]) |
| 28 | GQSAF[C(1)]I[1-Me-Trp]QDW[Sar]EHR[C(1)]TEGE[Peg3][Peg3]- |
| | K([17-Carboxy-heptadecanoyl][yGlu]G[yGlu]) |
| 29 | ESGVF[C(1)]I[1-Me-Trp]QDW[Sar]EHR[C(1)]TEGE[Peg3][Peg3]- |
| | K([17-Carboxy-heptadecanoyl][yGlu]G[yGlu]) |
| 30 | YEQAF[C(1)]I[1-Me-Trp]QDW[Sar]EHR[C(1)]TEGE[Peg3]ES-K([17- |
| | Carboxy-heptadecanoyl][yGlu]G[yGlu]) |
| 31 | SEFC(2)I[1-Me-Trp]QDWGEHRC(2)TGAES |

where [C(1)] indicates pairs of cysteine residues having a methylene bridging group between the sulphur atoms of their side chains, and [C(2)] indicates pairs of cysteine residues having an ethylene bridging group between the sulphur atoms of their side chains.

9. A compstatin analogue or pharmaceutically acceptable salt or solvate according to claim 8 selected from:

| | |
|---|---|
| 1 | Ac-SEF[C(1)]I[1-Me-Trp]QDWGEHR[C(1)]TGAES-[NH₂] |
| 2 | Ac-SEF[C(1)]I[1-Me-Trp]QDWGEHR[C(1)][Sar]EGE[Peg3][Peg3]- |
| | K([17-Carboxy-heptadecanoyl][yGlu]G[yGlu])-NH₂ |
| 3 | Ac-EGSAY[C(1)]I[1-Me-Trp]QDWGEHR[C(1)][Sar]EK[yGlu]-K([17- |
| | Carboxy-heptadecanoyl][yGlu][Peg3][Peg3])-NH₂ |
| 4 | Ac-SEF[C(1)]I[1-Me-Trp]QDWGEHR[C(1)][Sar]EGA-K([17-Carboxy- |
| | heptadecanoyl]-[yGlu]G[Peg3][yGlu][Peg3]-NH₂ |
| 5 | Ac-SEF[C(1)]I[1-Me-Trp]QDWGEHR[C(1)]TEGE[Peg3]ES-K([17- |
| | Carboxy-heptadecanoyl]-[yGlu]-NH₂ |
| 6 | Ac-GEF[C(1)]I[1-Me-Trp]QDWGEHR[C(1)][Sar]EAE[Peg3][Peg3]- |
| | K([17-Carboxy-heptadecanoy!][yGlu]G[yGlu])-NH₂ |
| 7 | Ac-SEF[C(1)]I[1-Me-Trp]QDW[Sar]EHR[C(1)][Sar]E[Peg3][Peg3]- |
| | K([17-Carboxy-heptadecanoy!][yGlu]G[yGlu])-NH₂ |
| 8 | Ac-SEF[C(1)]I[1-Me-Trp]QDW[Sar]EHR[C(1)][Sar]E[Peg3][Peg3]- |
| | K([17-Carboxy-heptadecanoyl]-[yGlu]G[yGlu]-NH₂ |
| 9 | Ac-SEF[C(1)]I[1-Me-Trp]QDW[Sar]EHR[C(1)]TEGE[Peg3][Peg3]- |
| | K([17-Carboxy-heptadecanoyl][yGlu]G[yGlu])-NH₂ |
| 10 | Ac-SEF[C(1)]I[1-Me-Trp]QDW[Sar]EHR[C(1)]TE[Peg3][Peg3]-K([17- |
| | Carboxy-heptadecanoy!][yGlu]G[yGlu])-NH₂ |
| 11 | Ac-SEF[C(1)]I[1-Me-Trp]QDW[Sar]EHR[C(1)]TEGE[Peg3]ES-K([17- |
| | Carboxy-heptadecanoyl][yGlu])-NH₂ |
| 12 | Ac-EF[C(1)]I[1-Me-Trp]QDW[Sar]EHR[C(1)]TEA[Peg3][Peg3]-K([17- |
| | Carboxy-heptadecanoyl][yGlu]G[yGlu])-NH₂ |
| 13 | Ac-SEF[C(1)]I[1-Me-Trp]QDW[SarJAHR[C(1)]TEGE[Peg3]ES-K([17- |
| | Carboxy-heptadecanoyl][yGlu])-NH₂ |
| 14 | Ac-SEF[C(1)]I[1-Me-Trp]QDWGEHR[C(1)]TGAES[Peg3][Peg3]- |
| | K([17-Carboxy-heptadecanoyl][yGlu]G[yGlu])-NH₂ |
| 15 | Ac-{d}YI[C(1)]I[1-Me-Trp]QDW[Sar]EHR[C(1)]TEGE[Peg3]ES-K([17- |
| | Carboxy-heptadecanoyl][yGlu])-NH₂ |
| 16 | Ac-SEF[C(1)]IWQDW[Sar]EHR[C(1)]TEGE[Peg3]ES-K([17-Carboxy- |
| | heptadecanoyl][yGlu])-NH₂ |
| 17 | Ac-SEF[C(1)]IYQDW[Sar]EHR[C(1)]TEGE[Peg3]ES-K([17-Carboxy- |
| | heptadecanoyl][yGlu])-NH₂ |
| 18 | Ac-SEY[C(1)]I[1-Me-Trp]QDW[Sar]EHR[C(1)]TEGE[Peg3]ES-K([17- |
| | Carboxy-heptadecanoyl][yGlu])-NH₂ |
| 19 | [15-Carboxy-pentadecanoyl]-EGSEF[C(1)]I[1-Me- |
| | TrpJQDW[Sar]EHR[C(1)]TEGE-[NH₂] |
| 20 | Ac-EGSEF[C(1)]I[1-Me-Trp]QDW[Sar]EHR[C(1)]TEGE[Peg3]ES- |
| | K([17-Carboxy-heptadecanoy!][yGlu])-NH₂ |
| 21 | Ac-KSGEF[C(1)]I[1-Me-Trp]QDW[Sar]EHR[C(1)]TEGE[Peg3][Peg3]- |
| | K([17-Carboxy-heptadecanoy][yGlu]G[yGlu])-NH₂ |
| 22 | Ac-EQEVF[C(1)]I[1-Me-Trp]QDW[Sar]EHR[C(1)]TEGE[Peg3][Peg3]- |
| | K([17-Carboxy-heptadecanoy!][yGlu]G[yGlu])-NH₂ |
| 23 | Ac-ESQVF[C(1)]I[1-Me-Trp]QDW[Sar]EHR[C(1)]TEGE[Peg3][Peg3]- |
| | K([17-Carboxy-heptadecanoyl][yGlu]G[yGlu])-NH₂ |
| 24 | Ac-ESEQVF[C(1)]I[1-Me- |
| | Trp]QDW[Sar]EHR[C(1)]TEGE[Peg3][Peg3]-K([17-Carboxy- |
| | heptadecanoyl][yGlu]G[yGlu])-NH₂ |
| 25 | Ac-SEQAF[C(1)]I[1-Me-Trp]QDW[Sar]EHR[C(1)]TEGE[Peg3][Peg3]- |
| | K([17-Carboxy-heptadecanoyl][yGlu]G[yGlu])-NH₂ |
| 26 | Ac-SKQEF[C(1)]I[1-Me-Trp]QDW[Sar]EHR[C(1)]TEGE[Peg3][Peg3]- |
| | K([17-Carboxy-heptadecanoyl][yGlu]G[yGlu])-NH₂ |
| 27 | Ac-EGESGF[C(1)]I[1-Me- |
| | Trp]QDW[Sar]EHR[C(1)]TEGE[Peg3][Peg3]-K([17-Carboxy- |
| | heptadecanoyl][yGlu]G[yGlu])-NH₂ |
| 28 | Ac-GQSAF[C(1)]I[1-Me-Trp]QDW[Sar]EHR[C(1)]TEGE[Peg3][Peg3]- |
| | K([17-Carboxy-heptadecanoyl][yGlu]G[yGlu])-NH₂ |
| 29 | H-ESGVF[C(1)]I[1-Me-Trp]QDW[Sar]EHR[C(1)]TEGE[Peg3][Peg3]- |
| | K([17-Carboxy-heptadecanoyl][yGlu]G[yGlu])-NH₂ |
| 30 | Ac-YEQAF[C()]I[1-Me-Trp]QDW[Sar]EHR[C()]TEGE[Peg3]ES-K([17- |
| | Carboxy-heptadecanoyl][yGlu]G[yGlu])-NH₂ |
| 31 | Ac-SEFC(2)I[1-Me-Trp]QDWGEHRC(2)TGAES-NH₂ |

where [C(1)] indicates pairs of cysteine residues having a methylene bridging group between the sulphur atoms of their side chains and [C(2)] indicates pairs of cysteine residues having an ethylene bridging group between the sulphur atoms of their side chains.

10. A compstatin analogue or pharmaceutically acceptable salt or solvate according to any one of claims 1 to 6 wherein the alkylene bridge is —CH₂— or —CH₂—CH₂—; that is, the linkage is —S—CH₂—S— or —S—CH₂—CH₂—S—.

11. A composition comprising a compstatin analogue or pharmaceutically acceptable salt or solvate according to any of the claims 1 to 10, in admixture with a carrier.

12. A composition according to claim 11, wherein the composition is a pharmaceutical composition and the carrier is a pharmaceutically acceptable carrier.

13. A pharmaceutical composition comprising a compstatin analogue or pharmaceutically acceptable salt or solvate according to any one of claims 1 to 10, in admixture with a pharmaceutically acceptable carrier, excipient or vehicle.

14. A compstatin analogue or pharmaceutically acceptable salt or solvate according to any one of claims 1 to 10 for use in therapy.

15. A compstatin analogue or pharmaceutically acceptable salt or solvate according to any one of claims 1 to 10 for use in a method of inhibiting complement activation.

16. The compstatin analogue or pharmaceutically acceptable salt or solvate for use according to claim 15, wherein inhibiting complement activation comprises one or more biological activities selected from (1) binding to C3 protein, (2) binding to C3b protein and/or (3) inhibiting the cleavage of native C3 by C3 convertases.

17. A compstatin analogue or pharmaceutically acceptable salt or solvate according to any one of claims 1 to 10 for use in a method of prophylaxis or treatment of age-related macular degeneration, Stargardt disease, periodontitis, diabetic retinopathy, glaucoma, uveitis, rheumatoid arthritis, spinal cord injury, stroke, multiple sclerosis, Parkinson's disease, Alzheimer's disease, cancer, and respiratory disorders such as asthma, chronic obstructive pulmonary disease (COPD), allergic inflammation, emphysema, bronchitis, bronchiecstasis, cystic fibrosis, tuberculosis, pneumonia, respiratory distress syndrome (RDS—neonatal and adult), rhinitis and sinusitis; bacterial infections such as sepsis, ischemia-reperfusion injury in various tissues, myocardial infarction, anaphylaxis, paroxysmal nocturnal hemoglobinuria, autoimmune hemolytic anemias, psoriasis, hidradentitis suppurativa, myasthenia gravis, systemic lupus erythematosus, CHAPLE syndrome, C3 glomeropathy, IgA nephropathy, atypical hemolytic uremic syndrome, Crohn's disease, ulcerative colitis or antiphospholipid syndrome.

18. A compstatin analogue or pharmaceutically acceptable salt or solvate according to any one of claims 1 to 10 for use in a method of inhibiting complement activation that occurs during cell or organ transplantation.

19. A method of inhibiting complement activation for treating a subject in need thereof, the method comprising administering to the subject a compstatin analogue or pharmaceutically acceptable salt or solvate according to any one of claims 1 to 10 to inhibit complement activation in the subject.

20. The method of claim 19, wherein the subject has age-related macular degeneration, Stargardt disease, periodontitis, diabetic retinopathy, glaucoma, uveitis, rheumatoid arthritis, spinal cord injury, stroke, multiple sclerosis, Parkinson's disease, Alzheimer's disease, cancer, and respiratory disorders such as asthma, chronic obstructive pulmonary disease (COPD), allergic inflammation, emphysema, bronchitis, bronchiecstasis, cystic fibrosis, tuberculosis, pneumonia, respiratory distress syndrome (RDS—neonatal and adult), rhinitis and sinusitis; bacterial infections such as sepsis, ischemia-reperfusion injury in various tissues, myocardial infarction, anaphylaxis, paroxysmal nocturnal hemoglobinuria, autoimmune hemolytic anemias, psoriasis, hidradentitis suppurativa, myasthenia gravis, systemic lupus erythematosus, CHAPLE syndrome, C3 glomeropathy, IgA nephropathy, atypical hemolytic uremic syndrome, Crohn's disease, ulcerative colitis or antiphospholipid syndrome.

21. An ex vivo method of inhibiting complement activation during extracorporeal shunting of a physiological fluid, the method comprising contacting the physiological fluid with a compstatin analogue or pharmaceutically acceptable salt or solvate according to any one of claims 1 to 10, thereby inhibiting complement activation.

22. Use of a compstatin analogue or pharmaceutically acceptable salt or solvate according to any one of claims 1 to 10, in the preparation of a medicament for inhibiting complement activation.

23. Use of a compstatin analogue or pharmaceutically acceptable salt or solvate according to any one of claims 1 to 10 in the preparation of a medicament for the treatment of age-related macular degeneration, Stargardt disease, periodontitis, diabetic retinopathy, glaucoma, uveitis, rheumatoid arthritis, spinal cord injury, stroke, multiple sclerosis, Parkinson's disease, Alzheimer's disease, cancer, and respiratory disorders such as asthma, chronic obstructive pulmonary disease (COPD), allergic inflammation, emphysema, bronchitis, bronchiecstasis, cystic fibrosis, tuberculosis, pneumonia, respiratory distress syndrome (RDS—neonatal and adult), rhinitis and sinusitis; bacterial infections such as sepsis, ischemia-reperfusion injury in various tissues, myocardial infarction, anaphylaxis, paroxysmal nocturnal hemoglobinuria, autoimmune hemolytic anemias, psoriasis, hidradentitis suppurativa, myasthenia gravis, systemic lupus erythematosus, CHAPLE syndrome, C3 glomeropathy, IgA nephropathy, atypical hemolytic uremic syndrome, Crohn's disease, ulcerative colitis or antiphospholipid syndrome.
